SEQUENCE LISTING (47 sequences)

SEQ ID NO: 1 - RNA, 20 nt, synthetic construct
  source 1..20: mol_type = other RNA
ccagaatggagaacgcagtg

SEQ ID NO: 2 - RNA, 18 nt, synthetic construct
  source 1..18: mol_type = other RNA
ccgtcaccaccacgaatt

SEQ ID NO: 3 - RNA, 38 nt, synthetic construct
  source 1..38: mol_type = other RNA
agcggtgaaccaagacgcagggcgcgatcaaaacaacg

SEQ ID NO: 4 - RNA, 41 nt, synthetic construct
  source 1..41: mol_type = other RNA
aattccctcgaggacaaggcgagctcttcggtagtagccaa

SEQ ID NO: 5 - RNA, 21 nt, synthetic construct
  source 1..21: mol_type = other RNA
ttattgggtaaaccttggggc

SEQ ID NO: 6 - RNA, 25 nt, synthetic construct
  source 1..25: mol_type = other RNA
tccaattaacaccaatagcagtcca

SEQ ID NO: 7 - RNA, 19 nt, synthetic construct
  source 1..19: mol_type = other RNA
tcattcgtttcggaagaga

SEQ ID NO: 8 - RNA, 23 nt, synthetic construct
  source 1..23: mol_type = other RNA
aggaactctagaagaattcagat

SEQ ID NO: 9 - RNA, 49 nt, synthetic construct
  source 1..49: mol_type = other RNA
tgtaactagcaagaataccacgaaacaggtacgttaatagttaatagcg

SEQ ID NO: 10 - RNA, 43 nt, synthetic construct
  source 1..43: mol_type = other RNA
gcttcgattgtgtgcgtactcgagagtaaacgtaaaaagaagg

SEQ ID NO: 11 - RNA, 24 nt, synthetic construct
  source 1..24: mol_type = other RNA
gctgcaatattgttaacgtgagtc

SEQ ID NO: 12 - RNA, 21 nt, synthetic construct
  source 1..21: mol_type = other RNA
acaaagccttacattaagtgg

SEQ ID NO: 13 - RNA, 25 nt, synthetic construct
  source 1..25: mol_type = other RNA
caccatcaacaaatatttttctcac

SEQ ID NO: 14 - RNA, 49 nt, synthetic construct
  source 1..49: mol_type = other RNA
tgggtggtatgtctgatcccaatagatttgttaaaatatgacttcacgg

SEQ ID NO: 15 - RNA, 43 nt, synthetic construct
  source 1..43: mol_type = other RNA
tgtgttaactgtttggatgacagattgtaagtgggaacactgt

SEQ ID NO: 16 - RNA, 25 nt, synthetic construct
  source 1..25: mol_type = other RNA
acggtcaaagagttttaacctctct

SEQ ID NO: 17 - RNA, 21 nt, synthetic construct
  source 1..21: mol_type = other RNA
gcattctgcattgtgcaaact

SEQ ID NO: 18 - RNA, 18 nt, synthetic construct
  source 1..18: mol_type = other RNA
tctttcacacgtggtgtt

SEQ ID NO: 19 - RNA, 19 nt, synthetic construct
  source 1..19: mol_type = other RNA
gtaccaaaaatccagcctc

SEQ ID NO: 20 - RNA, 46 nt, synthetic construct
  source 1..46: mol_type = other RNA
catggaaccaagtaacattggaaaacctgacaaagttttcagatcc

SEQ ID NO: 21 - RNA, 42 nt, synthetic construct
  source 1..42: mol_type = other RNA
ctctgggaccaatggtactaagaggacttctcagtggaagca

SEQ ID NO: 22 - RNA, 24 nt, synthetic construct
  source 1..24: mol_type = other RNA
ggtaagaacaagtcctgagttgaa

SEQ ID NO: 23 - RNA, 24 nt, synthetic construct
  source 1..24: mol_type = other RNA
gtttgataaccctgtcctaccatt

SEQ ID NO: 24 - RNA, 22 nt, synthetic construct
  source 1..22: mol_type = other RNA
ggtgtttattaccctgacaaag

SEQ ID NO: 25 - RNA, 19 nt, synthetic construct
  source 1..19: mol_type = other RNA
gtaccaaaaatccagcctc

SEQ ID NO: 26 - RNA, 50 nt, synthetic construct
  source 1..50: mol_type = other RNA
catggaaccaagtaacattggaaaattttcagatcctcagttttacattc

SEQ ID NO: 27 - RNA, 42 nt, synthetic construct; identical to SEQ ID NO: 21
  source 1..42: mol_type = other RNA

SEQ ID NO: 28 - RNA, 24 nt, synthetic construct
  source 1..24: mol_type = other RNA
gaaaggtaagaacaagtcctgagt

SEQ ID NO: 29 - RNA, 24 nt, synthetic construct
  source 1..24: mol_type = other RNA
gtttgataaccctgtcctaccatt

SEQ ID NO: 30 - RNA, 18 nt, synthetic construct
  source 1..18: mol_type = other RNA
agatcacattggcacccg

SEQ ID NO: 31 - RNA, 20 nt, synthetic construct
  source 1..20: mol_type = other RNA
ccattgccagccattctagc

SEQ ID NO: 32 - RNA, 41 nt, synthetic construct
  source 1..41: mol_type = other RNA
tgctcccttctgcgtagaagccaatgctgcaatcgtgctac

SEQ ID NO: 33 - RNA, 39 nt, synthetic construct
  source 1..39: mol_type = other RNA
ggcggcagtcaagcctcttccctactgctgcctggagtt

SEQ ID NO: 34 - RNA, 25 nt, synthetic construct
  source 1..25: mol_type = other RNA
ggcaatgttgttccttgaggaagtt

SEQ ID NO: 35 - RNA, 21 nt, synthetic construct
  source 1..21: mol_type = other RNA
cacgtagtcgcaacagttcaa

SEQ ID NO: 36 - RNA, 18 nt, synthetic construct
  source 1..18: mol_type = other RNA
agatcacattggcacccg

SEQ ID NO: 37 - RNA, 20 nt, synthetic construct
  source 1..20: mol_type = other RNA
ccattgccagccattctagc

SEQ ID NO: 38 - RNA, 41 nt, synthetic construct; identical to SEQ ID NO: 32
  source 1..41: mol_type = other RNA

SEQ ID NO: 39 - RNA, 39 nt, synthetic construct; identical to SEQ ID NO: 33
  source 1..39: mol_type = other RNA

SEQ ID NO: 40 - RNA, 24 nt, synthetic construct
  source 1..24: mol_type = other RNA
gcaatgttgttccttgaggaagtt

SEQ ID NO: 41 - RNA, 24 nt, synthetic construct
  source 1..24: mol_type = other RNA
gttcctcatcacgtagtcgcaaca

SEQ ID NO: 42 - RNA, 19 nt, synthetic construct
  source 1..19: mol_type = other RNA
tctattgccatacccacaa

SEQ ID NO: 43 - RNA, 19 nt, synthetic construct
  source 1..19: mol_type = other RNA
gtaccaaaaatccagcctc

SEQ ID NO: 44 - RNA, 50 nt, synthetic construct; identical to SEQ ID NO: 26
  source 1..50: mol_type = other RNA

SEQ ID NO: 45 - RNA, 42 nt, synthetic construct; identical to SEQ ID NO: 21
  source 1..42: mol_type = other RNA

SEQ ID NO: 46 - RNA, 24 nt, synthetic construct
  source 1..24: mol_type = other RNA
gaaaggtaagaacaagtcctgagt

SEQ ID NO: 47 - RNA, 24 nt, synthetic construct
  source 1..24: mol_type = other RNA
gtttgataaccctgtcctaccatt

(19) United States
(12) Patent Application Publication
(10) Pub. No.: US 2025/0236920 A1
(43) Pub. Date: Jul. 24, 2025

(54) METHOD OF DETECTING SARS-COV IN A SAMPLE

(71) Applicant: Imam Abdulrahman Bin Faisal University, Dammam (SA)

(72) Inventors: Huseyin TOMBULOGLU, Dammam (SA); Moneerah ALSAEED, Dammam (SA); Galyah ALHAMID, Dammam (SA)

(73) Assignee: Imam Abdulrahman Bin Faisal University, Dammam (SA)

(21) Appl. No.: 18/415,814

(22) Filed: Jan. 18, 2024

(51) Int. Cl. C12Q 1/70; C12Q 1/6844

(52) U.S. Cl. CPC C12Q 1/701; C12Q 1/6844; C12Q 2600/16

(57) ABSTRACT

A method of detecting SARS-CoV-2 includes contacting a sample with a primer set and reverse transcription loop-mediated isothermal amplification (RT-LAMP) reagents, which includes calcein, to form a reaction mixture, and amplifying the cDNA by incubating the reaction mixture to amplify the target sequence of the SARS-CoV-2 nucleic acid sequence in the sample. The sample is further assayed with an assay to detect the amplified target sequence of the SARS-CoV-2 nucleic acid sequence, and thereby to detect SARS-CoV-2 in the sample.

### STATEMENT OF ACKNOWLEDGEMENT

Support provided by Institute for Research and Medical Consultations (IRMC) under project number 2020-IRMC-S-4 and the Deanship of Scientific Research (DSR) of Imam Abdulrahman bin Faisal University (IAU) fund COVID19-2020-026-IRMC is gratefully acknowledged.

### REFERENCE TO SEQUENCE LISTING

In accordance with 37 CFR § 1.52(e)(5) and with 37 CFR § 1.831, the specification makes reference to a Sequence Listing submitted electronically as a .xml file named “548258US_final”. The .xml file was generated on Dec. 28, 2023 and is 42,421 bytes in size. The entire contents of the Sequence Listing are hereby incorporated by reference.

### BACKGROUND

### Technical Field

The present disclosure is directed to a method of detection of severe acute respiratory syndrome coronavirus (SARS-CoV-2) and particularly relates to a method for detecting the SARS-CoV-2 in a sample using reverse transcription loop-mediated isothermal amplification (RT-LAMP) and selected primers.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Coronavirus disease 2019, or COVID-19, was first reported in 2019 and soon led to a worldwide pandemic, causing more than a hundred million detected and undetected infections, widespread morbidity, and millions of deaths. COVID-19 has had a catastrophic effect on human lives the world over and has emerged as a major global health crisis. To contain the rapid increase and spread of COVID-19 infections caused by the SARS-CoV-2 virus, or COVID-19 virus, timely testing of symptomatic and asymptomatic persons has proven to be a globally effective measure.

Since the pandemic began, several advancements have been brought about for the diagnosis of the COVID-19 infection. Testing being at the forefront, two types of tests have been considered for early detection and diagnosis of the disease. First molecular tests such as those using the polymerase chain reaction (PCR) are used to detect viral nucleic acids in a biological sample; however, such tests often require sophisticated laboratory equipment and are typically conducted at dedicated testing sites. Such tests help determine the need for prevention measures, like isolation, and are useful for screening infected individuals.

Second, viral antigen tests detect surface antigens on the SARS-CoV-2 virus in a biological sample. In contrast with molecular tests, antigen tests swiftly detect the presence of specific viral proteins known as antigens, providing rapid results to determine current infections. However, such tests lack the sensitivity of molecular tests.

Another category of tests is antibody or serological testing, which are employed mainly for surveillance and determining epidemiological scenarios. The antibody testing detects specific antibodies that target specific parts of the virus. The antibody testing methodology is less useful for the detection of current infections. SARS-CoV-2 breath tests are under development which detect chemical compounds in the breath of a subject that are associated with SARS-CoV-2 infection.

While the availability of multiple testing methods is encouraging in a situation such as a pandemic, most of the methods mentioned above and presently utilized come with several drawbacks posing serious healthcare concerns. The most sensitive tests, such as PCR, require advanced equipment and have a long turnaround time. Certain equipment, such as a thermal cycler, is mandatory for performing the PCR. Also, a high percentage of false negative results have proved to be problematic for preferring the PCR method for COVID-19 infections. On the other hand, the antigen tests though rapid, produce a rate of false negative results is higher than the PCR tests. An additional concern regarding the serology immunoassays is the lack of accuracy to be a reliable SARS-CoV-2 diagnostic test. The tests also show low sensitivity and are less reliable.

Detection of SARS-CoV-2 suffers from other problems. The mutation rate of SARS-CoV-2 presents several challenges including reliable detection and epidemiological surveillance of SARS-CoV-2 and the emergence of variants that affect clinical responses against the virus. Mutation of SARS-CoV-2 decreases the sensitivity of existing tests, for example, by loss of detectable epitopes or genetic sequences.

An accurate and reliable diagnosis facilitates containment of COVID-19 and the identification of vaccines most suitable for treatment of COVID-19 caused by SARS-CoV-2 generally as well as for treatment of COVID caused by particular strains of SARS-CoV-2. Accurate diagnosis permits early identification and isolation of infected individuals, contact tracing, quarantine procedures, and other pandemic control measures. Accurate diagnosis is also a first step in developing or selecting drugs or vaccines that are effective against COVID-19 infection in general as well as those most effective against COVID-19 caused by SARS-CoV-2 variants.

As mentioned above, the current detection and diagnostic approaches suffer from low sensitivity and accuracy including production of false negative and false positive readings. Moreover, current approaches are labor intensive and slow due to time required for sample preparation. Current approaches also require the use of complex or cumbersome instruments and corresponding equipment, often at a high cost due to maintenance requirements and the employment of highly trained laboratory technicians.

As fast and early identification and treatment of individuals infected with SARS-CoV-2 is needed for control and treatment of COVID-19, there is a need for an accurate, reliable, broadly applicable to subjects infected with SARS-CoV-2 and its variants, user-friendly, rapid, robust, scalable, and cost-effective method for detecting subjects infected by SARS-CoV-2. As disclosed herein, the inventors have developed such a method and associated primer compositions for visual detection of SARS-CoV-2 using loop-mediated isothermal amplification of SARS-CoV nucleic acids.

### SUMMARY

In one embodiment, a method of detecting SARS-CoV-2 in a sample using a primer set selected from the group consisting of N-ID5 (Set-1), E-ID1 (Set-2), RdRp-ID37 (Set-3), S-ID17 (Set-4), S-ID24 (Set-5), N-ID15 (Set-6), and N-ID15n1L (Set-7) is disclosed. Typically, this method comprises contacting a sample suspected of containing SARS-CoV-2 with the above-mentioned primer set and with one or more reverse transcription loop-mediated isothermal amplification (RT-LAMP) reagents to form a reaction mixture that includes calcein.

The reaction mix is incubated at a temperature and for a time sufficient to amplify a target sequence defined by the primers in SARS-CoV-2 nucleic acid sequence in the sample.

The method includes detecting the presence or amount of the amplified target sequence by assaying the sample with an assay to detect the amplified target sequence of the SARS-CoV-2 nucleic acid sequence for detecting a presence of the amplified target sequence of the SARS-CoV-2 nucleic acid sequence, thereby detecting SARS-CoV-2 in the sample.

In another embodiment a method of detecting SARS-CoV-2 in a sample using a primer set S-set11 (Set-8) is disclosed. The method includes contacting the sample with a primer set and one or more reverse transcription loop-mediated isothermal amplification (RT-LAMP) reagents to form a reaction mixture. The RT-LAMP reagents comprise calcein. The reaction mixture is incubated at a temperature and for a time sufficient to amplify a target sequence of the SARS-CoV-2 nucleic acid sequence in the sample. The method includes detecting the presence or amount of the amplified target sequence by assaying the sample with an assay to detect the amplified target sequence of the SARS-CoV-2 nucleic acid sequence and detecting a presence of the amplified target sequence of the SARS-CoV-2 nucleic acid sequence, thereby detecting SARS-CoV-2 in the sample.

In some embodiments, the RT-LAMP reagents includes at least one Bst deoxyribose nucleic acid (DNA) polymerase, deoxyribonucleotide triphosphates (dNTPs), magnesium ions, a buffer, a guanidine hydrochloride, and calcein.

In some embodiments, the amplifying is a reverse-transcription loop-mediated isothermal amplification (RT-LAMP) process.

In some embodiments, the method includes extracting nucleic acid from the sample.

In some embodiments, the assay is a colorimetric RT-LAMP assay.

In some embodiments, the assaying includes mixing a master mix, a primer mix, the sample, calcein, and dH₂O to form a colorimetric RT-LAMP mixture, and incubating the colorimetric RT-LAMP mixture in a water bath at a constant temperature for a time.

In some embodiments, incubating the reaction mixture occurs for 20 to 70 minutes.

In some embodiments, the primer set is N-ID5 and a limit of detection of the assay is 0.01 to 0.2 copies/μL of the sample.

In some embodiments, the SARS-CoV-2 is SARS-CoV-2, and the SARS-CoV-2 includes at least one variant selected from the following group consisting of alpha (B.1.1.7), beta (B.1.351), gamma (P.1), delta (B.1.617.2), and omicron (B.1.1.529).

In some embodiments, the target sequence of the SARS-CoV-2 nucleic acid sequence is located within at least one gene selected from the group consisting of nucleocapsid (N), spike (S), RNA-dependent RNA polymerase (RdRp), and envelope (E) of a SARS-CoV-2 genome.

In some embodiments, detecting the presence of the amplified target sequence is observed with a visual color change, a transilluminator, and an ultraviolet light.

In some embodiments, an average time to react (TTR) is from 20 to 60 minutes.

In some embodiments, the primer set is N-ID5, and the detection of the presence of the amplification product for the target sequence of a SARS-CoV-2 nucleic acid sequence includes an accuracy of at least 94% for the colorimetric RT-LAMP assay. The accuracy is based on colorimetric RT-LAMP assay results compared to reverse transcription real-time polymerase chain reaction (RT-qPCR) assay results of the sample with a sample size of at least 65 samples.

In some embodiments, the primer set is N-ID5, and the assay has a specificity of at least 99% for the colorimetric RT-LAMP assay. The specificity is based on colorimetric RT-LAMP assay results compared to RT-qPCR assay results of the sample with a sample size of at least 65 samples.

In some embodiments, the primer set is N-ID5, and the assay has a sensitivity of at least 90% for the colorimetric RT-LAMP assay. The sensitivity is based on colorimetric RT-LAMP assay results compared to RT-qPCR assay results of the sample with a sample size of at least 65 samples.

In some embodiments, the primer set is N-ID5 and a positive-percent agreement of the RT-LAMP assay of the sample is at least 99% based on a true positive and a false positive when compared to real-time reverse-transcriptase polymerase chain reaction (RT-qPCR) assay results of the sample with a sample size of at least 65 samples.

In some embodiments, the primer set is N-ID5 and a negative-percent agreement of the RT-LAMP assay of the sample is at least 85% based on a true negative and a false negative when compared to real-time reverse-transcriptase polymerase chain reaction (RT-qPCR) assay results of the sample with a sample size of at least 65 samples.

In some embodiments, the method further includes treating a subject for which the sample was obtained.

In another exemplary embodiment, a kit for detection of SARS-CoV-2 in a sample is described. The kit includes a Bst polymerase, calcein, a universal primer set for loop-mediated isothermal amplification (LAMP) of a target sequence in a SARS-CoV-2 nucleic acid sequence and variants thereof containing mutations within one or more primer binding sites. The universal primer set suitable for LAMP hybridizes to the target sequence of a SARS-CoV-2 nucleic acid sequence in the presence of a plurality of undefined mutations and is configured to provide a positive result for the target sequence of a SARS-CoV-2 nucleic acid sequence in a predetermined assay time period otherwise determined for a positive sample of a target nucleic acid having a known sequence. Any of the reagents in the kit may be combined in a mixture in a single container or provided in separate containers.

The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of the present disclosure (including alternatives and/or variations thereof) and many of the attendant advantages thereof will be readily obtained as the same becomes better understood by reference to the following detailed description of the embodiments when considered in connection with the accompanying drawings, wherein:

FIG. 1 is a flow chart depicting a method of detecting SARS-CoV-2 in a sample, according to certain embodiments;

FIG. 2A depicts the binding position of six loop-mediated isothermal amplification (LAMP) primers, namely LB, LF, FIP, BIP, F3, and B3, and the formation of stem-loop and dumbbell structures, which serve as starting points for the LAMP amplification cycles, according to certain embodiments;

FIGS. 2B-2C depict the input and output of the LAMP reaction upon the activation of the Bst enzyme, according to certain embodiments;

FIG. 3A depicts observation of calcein-based LAMP results of a positive control (PC) and a negative control (NC) sample by the natural eye, according to certain embodiments;

FIG. 3B depicts observation of calcein-based LAMP results of PC and NC samples by ultraviolet (UV) light, according to certain embodiments;

FIG. 3C depicts a hand UV instrument and protective glasses, according to certain embodiments;

FIG. 3D depicts observation of calcein-based LAMP results of PC and NC samples under a transilluminator, according to certain embodiments;

FIG. 3E shows the detection of SARS-CoV-2 in the positive control (PC) and negative control (NC) tubes by using different LAMP primer sets (N-ID5, E-ID1, S-set11, RdRp-ID37, S-ID17, S-ID24, N-ID15, and N-ID15n1L) by the natural eye, according to certain embodiments;

FIG. 3F shows the average detection time of calcein-based LAMP using different primer sets (N-ID5, E-ID1, S-set11, RdRp-ID37, S-ID17, S-ID24, N-ID15, and N-ID15n1L), according to certain embodiments;

FIG. 4A shows the effect of Bst3.0 enzyme concentration (1×, 2×, and 3×) on color development and the detection of SARS-CoV-2 using N-ID5 primers, as observed by the natural eye (visual), transilluminator, and UV light, according to certain embodiments;

FIGS. 4B-4D are pictorial images depicting RGB (red, green, and blue) color intensities that were gradually increased by increasing enzyme concentration, according to certain embodiments;

FIG. 5A shows clinically verified SARS-CoV-2 positive (P1-P8), samples observed by a transilluminator, the natural eye (visual), and UV light, according to certain embodiments;

FIG. 5B shows clinically verified SARS-CoV-2 negative (N1-N6), samples observed by using a transilluminator, the natural eye (visual), and UV light, according to certain embodiments;

FIG. 5C shows the RGB, Lab, and hue, saturation, brightness (HSB) values determined using a mobile application (Color Wheel Analyzer, CWA) in PC and NC samples, according to certain embodiments;

FIG. 5D depicts the mean RGB, Lab, and HSB codes in PC and NC samples, according to certain embodiments;

FIGS. 5E-5G depicts the mean RGB, Lab, and HSB codes in PC and NC samples that were observed visually, according to certain embodiments;

FIGS. 5H-5J depicts mean RGB, Lab, and HSB codes in PC and NC samples that were observed by a transilluminator, according to certain embodiments;

FIGS. 5K-5M depicts mean RGB, Lab, and HSB codes in PC and NC samples that were observed by a UV light, according to certain embodiments;

FIG. 6A is a depiction of the detection limit of calcein-based colorimetric RT-LAMP assay of ribonucleic acid (RNA) sample of SARS-CoV-2 (Delta variant) with an unknown concentration that was serially diluted (1, 10¹, 10², 10³, 10⁴, and 10⁵-fold), according to certain embodiments;

FIG. 6B is a depiction of the detection limit of calcein-based colorimetric RT-LAMP assay of known concentration of pseudo viral SARS-CoV-2 RNA (Twist Bioscience, Omicron variant) that was serially diluted (1, 10¹, 10², 10³, 10⁴, 10⁵, and 10⁶-fold), according to certain embodiments;

FIG. 6C shows target sequences of the F3 primer on the genome of Delta and Omicron variants, according to certain embodiments; and

FIG. 6D shows the average time-to-reaction (TTR) value of calcein-based RT-LAMP assay (minutes) (n=53), according to certain embodiments.

### DETAILED DESCRIPTION

In the following description, it is understood that other embodiments may be utilized, and structural and operational changes may be made without departure from the scope of the present embodiments disclosed herein.

Reference will now be made in detail to specific embodiments or features, examples of which are illustrated in the accompanying drawings. Whenever possible, corresponding or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

The term “master mix” refers to a combination of reagents which can be added to a sample to execute a reaction in an assay where the combination enhances the efficiency and speed of performing the assay. The master mix described herein may include reagents to reduce non-specific amplification. The master mix may include a strand displacing DNA polymerase and may additionally include other enzymes such as a reverse transcriptase. The master mix may also include a reversible inhibitor of DNA polymerase activity. An example of a reversible inhibitor is an oligonucleotide known as an aptamer that binds to the DNA polymerase and blocks its activity below a selected temperature (for example, 50, 55, or 60° C.) but above that temperature, the oligonucleotide is disassociated from the enzyme, permitting the reverse transcriptase to become active. In some embodiments, the master mix includes a reverse transcriptase and a reversible inhibitor of reverse transcriptase activity for inhibiting the activity of these enzymes below 40, 45, or 50° C. An example of a reversible inhibitor is an oligonucleotide known as an aptamer that binds to the reverse transcriptase and blocks activity below a certain temperature (for example, 40° C.) but above that temperature, the oligonucleotide is disassociated from the enzyme, permitting the reverse transcriptase to become active. This allows setting up a reaction at room temperature while avoiding non-specific amplification. The master mix may also comprise inhibitors of nucleases such as RNase inhibitors and/or DNase inhibitors. These inhibitors may be chemical reagents such as poloxamers, and/or aptamers. However, nucleases may also be inactivated by submitting the sample combined with a lysis buffer to a high temperature for an effective time. The master mix may also include dNTPs such as dTTP, dATP, dGTP, and dCTP as well as dUTP for carryover prevention. For example, a 2× master mix may contain the dNTPs in equal quantities except the dUTP at 50% concentration of the other dNTPs. The master mix may include single strand binding proteins and/or helicases to reduce non-specific amplification. The master mix may also include a dye that can be a pH-sensitive or metallochromic dye. The master mix includes a fluorescent dye, calcein.

The master mix may be lyophilized or freeze-dried. It may be preserved for storage in a suitable buffer that may contain at least one reducing agent and at least one buffer that may contain at least one reducing agent and at least one detergent capable of storage at −20° C. for an extended period (for example, months). The inclusion of a reducing agent may be desirable if RNA is the template nucleic acid.

The master mix may be prepared in a 2×, 3×, 4×, 5×, 10×, or any suitable concentration. The master mix, once diluted by the sample, will result in a 1× concentration. The master mix may contain primers, or primers are not contained in the master mix.

As used herein, the term “primer” refers to a short single-stranded oligonucleotide sequence complementary to a nucleic acid strand to be replicated. It may serve as a starting point for synthesizing a primer extension product. It may act as a point of initiation of synthesis when placed under conditions in which synthesis of primer extension product, which is complementary to a nucleic acid strand (template), is induced and/or in the presences of nucleotides and an agent for polymerization, such as DNA polymerase, and at a suitable temperature and pH.

As used herein, the term “oligonucleotide(s)” refers to short DNA and/or RNA molecules, oligomers or oligos, used in a range of genetic testing and research. Oligonucleotides may be synthesized in a laboratory by solid-phase chemical synthesis and may be single-stranded molecules with any user-specified nucleic acid sequence. Oligonucleotides may be used for artificial gene synthesis, polymerase chain reaction (PCR), DNA sequencing, molecular cloning, as molecular probes, and the like. Oligonucleotides bind in a sequence-specific manner, to their respective complementary oligonucleotides, DNA, or RNA to form duplexes or, less often, hybrids of a higher order. Examples of procedures that use oligonucleotides include, but are not limited to, DNA microarrays, Southern blots, fluorescent in situ hybridization (FISH), PCR, the synthesis of artificial genes, and any procedure known in the art.

As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases capable of hybridizing to at least one nucleic acid strand even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single stranded nucleic acid molecule.

Aspects of the present disclosure are directed to a method of detecting SARS-CoV-2 in a sample, such as a clinical sample. Such detection may be accomplished in situ or in vitro but is preferably conducted in vitro. As used herein, “sample” refers to any material or mixture of materials containing one or more analytes or entities of interest. As used herein, “analyte” refers to any substance that is suitable for testing in the present disclosure. The sample that may be evaluated in accordance with the present disclosure include any that may contain SARS-CoV-2 and can be derived from a human patient or an animal, e.g., bodily fluids (blood, nasal secretions, saliva, urine, and the like), biopsy, tissue, and/or waste from the patient. Thus, tissue biopsies, stool, sputum, saliva, blood, lymph, tears, sweat, urine, nasal secretions, or the like can be used in the method, as can any tissue of interest. Preferably, however, the employed sample will be a nasal swab sample, a nasopharyngeal swab sample, a pharyngeal swab sample, or a sputum sample, and most preferably, the employed clinical sample will be a nasopharyngeal swab sample. In an embodiment, the sample may be pretreated to extract RNA that may be present in the sample. Alternatively, and more preferably, the sample will be evaluated without prior RNA extraction.

The method includes contacting the sample with a primer set and one or more reverse transcription loop-mediated isothermal amplification (RT-LAMP) reagents to form a reaction mixture. The RT-LAMP reagents comprise calcein. Calcein, also known as fluorexon and/or fluorescein complex, is a fluorescent dye with an excitation wavelength of 495 nm and an emission wavelength of 515 nm. Calcein has an orange appearance in solution when bound with certain metal ions such as magnesium. Calcein appears green in solution when bound with metal ions such as manganese.

As used herein, the term “oligonucleotide primer(s)” denotes a nucleic acid molecule that comprises at least 10 nucleotide residues and not more than 500 nucleotide residues, more preferably, not more than 200 nucleotide residues, still more preferably, not more than 100 nucleotide residues, and still more preferably, not more than 50 nucleotide residues, and that is capable of specifically hybridizing to the target sequence. Typically, an oligonucleotide contains a 5′ phosphate at one terminus (“5′ terminus”) and a 3′ hydroxyl group at the other terminus (“3′ terminus”) of the chain. The most 5′ nucleotide of an oligonucleotide may be referred to herein as the “5′ terminal nucleotide” of the oligonucleotide. The most 3′ nucleotide of an oligonucleotide may be referred to herein as the “3′ terminal nucleotide” of the oligonucleotide. As used herein, the term “hybridize” refers to a process of formation of double stranded nucleic acid regions between one, two, or many single stranded nucleic acid molecules complementary to one, two, or many target nucleic acid sequences. Hybridization may occur through specific hydrogen bonds between standard (Watson-Crick) base pair(s). Nucleic acid(s) that are “complementary” or “complement(s)” are those that are capable of base-pairing according to the standard Watson-Crick, Hoogsteen, or reverse Hoogsteen binding complementarity rules. As used herein, the term “specifically hybridizing” denotes the capability of a nucleic acid molecule to detectably anneal to another nucleic acid molecule, preferably a complementary nucleic acid molecule, under conditions in which such nucleic acid molecule does not detectably anneal to a non-complementary nucleic acid molecule.

As used herein, the term “nucleic acid,” includes both deoxyribonucleic acid (DNA) and ribonucleic acid (RNA), including DNA and RNA containing non-standard nucleotides. A “nucleic acid” contains at least one polynucleotide (a “nucleic acid strand”). The term “nucleic acid,” as used herein, refers to either deoxyribonucleic acid (DNA), ribonucleic acid (RNA), single-stranded or double-stranded, and any chemical modifications thereof. The nucleic acid detected according to the systems, assays, and/or methods disclosed herein may be a full-length nucleic acid or a fragment thereof. Nucleic acids are biopolymers and macromolecules comprising nucleotides. Nucleotides are monomer components comprising a five-carbon sugar, a phosphate group, and a nitrogenous base. The nitrogenous base, or nucleobase, may be adenine “A”, cytosine “C”, guanine “G”, thymine “T”, uracil “U”, and any nitrogenous base known in the art. The primer set may comprise one or more primers. The primers may comprise one or more oligonucleotide primers. In an embodiment, the primer set is selected from the group consisting of N-ID5 (Set-1), E-ID1 (Set-2), RdRp-ID37 (Set-3), S-ID17 (Set-4), S-ID24 (Set-5), N-ID15 (Set-6), and N-ID15n1L (Set-7).

In some embodiments, the RT-LAMP reagents may comprise one or more fluorescent dyes in addition to calcein. In some embodiments, the RT-LAMP reagents may comprise one or more fluorescent dyes in place of calcein. The one or more fluorescent dyes may be a Fura-2, a Furaptra, a Indo-1, an aequorin, and/or any fluorescent dye known in the art suitable for use in RT-LAMP such as dyes binding to single-stranded or double-stranded DNA or RNA and/or to metal ions bound or complexed to DNA or RNA, such as magnesium, calcium, cobalt, zinc, iron, copper, and any metal ions known in the art.

In some embodiments, the one or more fluorescent dyes may bind magnesium in order to fluoresce. In other embodiments, the one or more fluorescent dyes may bind any other metal ion known in the art to fluoresce. In some embodiments, the RT-LAMP reagents may comprise one or more non-fluorescent dyes in addition to calcein. In some embodiments, the RT-LAMP reagents may comprise one or more non-fluorescent dyes in place of calcein. Advantageously, the methods disclosed herein are performed with reagents comprising calcein.

As used herein, a “polynucleotide” refers to a polymeric chain containing two or more nucleotides, which contain deoxyribonucleotides, ribonucleotides, and/or their analog, such as those containing modified backbones (e.g., peptide nucleic acids (PNAs) or phosphorothioates) or modified bases. Polynucleotides includes primers, oligonucleotides, nucleic acid strand, and the like. A polynucleotide may contain standard or non-standard nucleotides. Thus, the term includes mRNA, tRNA, rRNA, ribozymes, DNA, cDNA, recombinant nucleic acids, branched nucleic acids, plasmids, vectors, probes, primers, and the like. A “nucleic acid” may be single-stranded or double-stranded. The term “nucleic acid” refers to nucleotides and nucleosides which make up, for example, DNA macromolecules and RNA macromolecules. The most common nucleic acids are DNA and RNA. In some embodiments, the target sequence is DNA or RNA.

In some embodiments, the method may optionally include reverse transcribing the target sequence of a SARS-CoV nucleic acid sequence in the sample into complementary DNA (cDNA) prior to or concurrently with addition of RT-PCR primers. Thus, reverse transcription of RNA to cDNA may occur as a one step or two step process.

Reverse transcription may take an RNA sequence, for example the target sequence of the SARS-CoV-2 nucleic acid sequence, and synthesis a double-stranded DNA product. This process may be done with the use of one or more RNA-dependent DNA polymerases, known as reverse transcriptases.

The method includes incubating the reaction mixture at a temperature and for a time sufficient to amplify the target sequence of the SARS-CoV-2 nucleic acid sequence in the sample. In some embodiments, the method may optionally include amplifying the cDNA by incubation. As used herein, the term “amplifying” or “amplification” denotes a process by which the number of copies of a gene is increased without a proportional increase in other genes. In some embodiments, the temperature may be 40 to 90° C., preferably 50 to 80° C., more preferably 60 to 70° C., and yet more preferably about 63° C. In some embodiments, the time may be 5 to 120 minutes, preferably 10 to 100 minutes, preferably 15 to 80 minutes, preferably 20 to 60 minutes, preferably 25 to 40 minutes, or preferably up to about 60 minutes.

The method further comprises assaying the sample with an assay to detect the amplified target sequence of a SARS-CoV-2 nucleic acid sequence.

The method further includes detecting a presence of the amplified target sequence of the SARS-CoV-2 nucleic acid sequence, thereby detecting SARS-CoV-2 in the sample. Detecting SARS-CoV-2 in the sample may be any positive assay result. Detecting SARS-CoV-2 in the sample may be a result of a change in color that may be visible to a human eye or with the aid of a spectrophotometer such as an ultraviolet-visible spectrophotometer and/or a transilluminator, a change in fluorescence, a combination thereof, and a change in any other signal known in the art.

In an embodiment, the method comprises contacting the sample with a primer set and the primer set is S-set11 (Set-8).

In an embodiment, the RT-LAMP reagents comprise at least one Bst (Bacillus stearothermophilus) deoxyribose nucleic acid (DNA) polymerase, deoxyribonucleotide triphosphates (dNTPs), magnesium ions, a buffer, a guanidine hydrochloride, and calcein. In some embodiments, the RT-LAMP reagents may comprise any RT-LAMP reagents known in the art. In an embodiment, Bst DNA polymerase is a reverse transcriptase and/or has reverse transcriptase activity. In some embodiments, the RT-LAMP reagents may comprise one or more reverse transcriptases.

In some embodiments, the amplifying is loop-mediated isothermal amplification (LAMP) and, more specifically, reverse-transcription loop-mediated isothermal amplification (RT-LAMP). LAMP is a nucleic acid amplification method that relies on auto-cycle strand-displacement DNA synthesis performed by Bst DNA polymerase, or other strand displacement polymerases. In LAMP, four primers recognize six unique target sequences on the template strand. Two of the primers are designated as “inner primers” (FIP and BIP) and two are designated as “outer primers” (F3 and B3). The reaction is initiated by annealing and extension of a pair of loop-forming primers (FIP and BIP), followed by annealing and extension of a pair of flanking primers (F3 and B3). Extension of these primers results in strand-displacement of loop-forming elements, which fold up to form hairpin-loop structures. In addition to containing a sequence that is complementary to a target sequence at their 3′ ends, the inner primers also contain a tail that comprises a sequence that is downstream of the 3′ end of primers in the template. Thus, an extension of an inner primer results in a product that has a self-complementary sequence at the 5′ end. Displacement of this product by an outer primer generates a product that has a loop at the 5′ end. Thus, the primer sets used in LAMP typically contain four, five, or six template-complementary sequences, where four sequences are found at the 3′ ends of the primers and two of the sequences are found at the 5′ ends of the primers. The initial reaction in LAMP results in a DNA product that has a dumbbell-like structure. In this product, the ends form stem loops and the single stranded region in between the stem loops is copied from the template. This product self-primes its own amplification to amplify the template sequence. LAMP uses a strand-displacing polymerase, and is isothermal, that is, it does not require heating and cooling cycles. The amplification process proceeds in an exponential manner (rather than a cyclic manner, like PCR) until all of the nucleotides (dATP, dTTP, dCTP dGTP, dUTP, and the like) in the reaction mixture have been incorporated into the amplified DNA. Optionally, an additional pair of primers may be included to accelerate the reaction. These primers, termed loop primers (LF and LB), hybridize to non-inner primer bound terminal loops of the inner primer dumbbell shaped products. Applications for LAMP have been further extended to include detection of RNA molecules by addition of reverse transcriptase (RT) enzyme. By including RNA detection, the types of targets for which LAMP can be applied are also expanded and add the ability to additionally target RNA based viruses, regulatory non-coding RNA (sRNA, miRNA), and RNA molecules that have been associated with particular disease or physiological states. The ability to detect RNA also has the potential to increase assay sensitivity, for instance, in choosing a highly expressed, stable, and/or abundant messenger RNA (mRNA) or ribosomal RNA (rRNA) targets. The preliminary phase of amplification involves the reverse transcription of RNA molecules to complementary DNA (cDNA). The cDNA then serves as template for the strand displacing DNA polymerase. Use of a thermostable RT enzyme enables the reaction to be completed at a single temperature and in a one step, single mix reaction.

RT-LAMP is a nucleic acid amplification method that relies on the addition of a reverse transcriptase, such as RTx Reverse Transcriptase or other reverse transcriptase enzymes, to the LAMP reaction to detect RNA target sequences. The amplified products are stem-loop structures with several repeated sequences of the target sequence and have multiple loops. The merit of this method is that denaturation of the DNA template is not required, and the LAMP and RT-LAMP reactions can occur under isothermal conditions (ranging from 35° C. to 75° C.).

LAMP is characterized by the use of four different primers specifically designed to recognize six distinct regions of the target gene. The four primers include two inner primers (forward inner primer (FIP) and backward inner primer (BIP)) and two out primers (forward outer primer (F3) and backward outer primer (B3)). A universal primer set may also include two additional primers, known as loop primers (loop forward (LF) and loop backward (LB)) primers. Although loop primers are optional, they may increase sensitivity and specificity of the amplification reaction. LAMP requires one enzyme and four to six primers that recognize six to eight distinct hybridization sites in the target sequence. The reaction can be accelerated with the addition of two additional primers, for a total of six primers. RT-LAMP requires two enzymes and four to six primers that recognize six to eight distinct hybridization sites in the target sequence. The method produces a large amount of amplified product, resulting in easier detection, such as detection by fluorescence or color of the reaction mixture. In some examples of the above embodiments, the RT-LAMP detects the target sequence in the sample and reverse transcribes the target sequence to a cDNA strand. In one embodiment, the amplification is detected involving a change in color or fluorescence visible to the natural (naked) eye. In some embodiments, a change of color is measured via a visual color change, a transilluminator, and an ultraviolet visible light. As used herein, “fluorescence” refers to the emission of light by a substance that has absorbed light or other electromagnetic radiation and may include luminescence, photoluminescence, fluorescence, and phosphorescence.

As used herein, the terms “sensitivity” and “specificity” as used herein have the meaning commonly understood in the art. Percent specificity is a measure of how well a test can identify true negatives in the sample. Specificity, or a true negative rate, is a measure of the probability of a negative test result, conditioned on the sample truly being negative. For example, if the biosensor of the present disclosure has a 100% specificity towards SARS-CoV-2 virus, the biosensor would detect no false positive cases, i.e., samples without a target sequence of SARS-CoV-2 would not be falsely identified as containing a target sequence of SARS-CoV. For further reference on specificity, see T. Fawcett, “An introduction to ROC analysis,” 2006, 27, 8, 861-874, incorporated herein by reference in its entirety.

In some examples, the target sequence is an RNA sequence of the SARS-CoV-2 virus present in the sample. A “target sequence,” as used herein, refers to a nucleic acid sequence of the SARS-CoV-2 genome, or a compliment thereof, that is amplified, detected, or both amplified and detected using one or more oligonucleotides herein provided. Additionally, while the term target sequence sometimes refers to a double stranded nucleic acid sequence, those skilled in the art will also recognize that the target sequence can also be single stranded, e.g., RNA, as is the case herein. As used herein, the terms “target nucleic acid” or “target” refer to a nucleic acid sequence to be analyzed or detected, e.g., a nucleic acid to which a probe hybridizes. The term refers to both the subsequence to which the probe is directed and to a larger nucleic acid sequence, including the subsequence. In an embodiment, the target sequence may be a “target nucleic acid” which, as used herein, refers to any nucleic acid that is targeted by primers to determine its presence in a sample. A target sequence may be selected that is more or less specific to an entire genus, to more than one genus, to a species or subspecies, serogroup, auxotype, serotype, strain, isolate or other subset of organisms.

RT-LAMP assays are described herein that enable rapid and sensitive detection of target nucleic acids, such as a nucleic acid of, or associated with, a pathogen, such as a virus, in a human or animal population. These assays are simple and portable while retaining sensitivity and minimizing false positives and negatives. The RT-LAMP assays described herein rely on detection of a change in some aspect, such as fluorescence, or color of a dye in a reaction mix due to a change in pH or metal ion. Turbidity may also be used as an end point in some RT-LAMP assays. In some embodiments, detection of the RT-LAMP amplified products can be achieved by a change in turbidity, colorimetrics, fluorescence, and/or any detection methods known in the art.

In some embodiments, the method further includes extracting nucleic acid from the sample. As used herein, the term “extracting” means obtaining, removing, or taking out with effort. In some embodiments, the extracting may include a separate protocol and a separate kit. The extracting may include disruption of a biological sample, including a tissue and/or cellular structure, denaturation of nucleoprotein complexes, inactivation of nucleases, and/or nucleic acid purification. In some embodiments, the extracting may be modified depending on the nucleic acid of interest and the sample source.

Detection of the RT-LAMP amplified products can be achieved via a variety of methods. In a preferred embodiment, the assay is a colorimetric RT-LAMP assay. As used herein, the term “colorimetric test(s)” refers to detection of a target nucleic acid by a change in color of a dye in a reaction mix due to a metal ion binding and/or dissociating. The assaying comprises mixing a master mix, a primer mix, the sample, calcein, and dH₂O to form a colorimetric RT-LAMP mixture; and incubating the colorimetric RT-LAMP mixture in a water bath at a constant temperature for a time. In some examples, the RT-LAMP mixture is incubated at a temperature between 50 to 70° C. until the color change is visible. In some embodiments, the color change is from orange and/or yellow to green and is visible by the natural eye. In some embodiments, the preferred temperature for the incubation is 63° C., and the preferred time for incubation is 20 to 70 minutes, preferably less than 60 minutes.

Aspects of the present disclosure are directed to colorimetric detection of SARS-CoV-2 using reverse transcription loop-mediated isothermal amplification (RT-LAMP). The RT-LAMP technique allows rapid and visual observation without the need for expensive instrumentation, such as quantitative thermal cyclers, as used in qPCR. Dye selection in colorimetric RT-LAMP acquires assay sensitivity and performance. A series of primers were designed, and the performance of RT-LAMP assay for the colorimetric detection of SARS-CoV-2 using fluorometric dye, namely calcein, was investigated. The assay performance is compared with RT-qPCR results on 67 clinical samples. The detection limit (LoD) with the N-ID5 primer set results in a performance corresponding to ˜2 copies/reaction or ˜0.1 copies/microliters (μL) of the RNA sample.

The diagnostic LAMP methods described herein may be used for detecting pathogens, including any of: prokaryotes such as bacteria, eukaryotic pathogens such as multicellular parasites, fungi, single cell pathogen such as trypanosomes or yeasts, and mycoplasma; as well as for use in genetic diseases, and in personalized medicine, which may require SNP detection or gene analysis of the genome of a subject or RNA analysis to determine gene expression profiles in response to an environmental or metabolic event. Other diagnostic uses include testing food for undesirable biological entities and monitoring environmental samples which may include biological material and are here referred to as biological samples also. The examples provided herein are directed to the SARS-CoV-2 RNA virus; however, the method described herein may be applied to any colorimetric or fluorescent LAMP-based assay to detect a target nucleic acid and may find applications in non-LAMP assays as well.

Compared to RT-qPCR, this assay allows sensitive and visual detection of SARS-CoV-2, with a high sensitivity, selectivity, and accuracy. The assay is sensitive regardless of variants, including delta and omicron. Since this assay uses an intercalating dye, calcein, for visual observation, it can be easily adapted in RT-LAMP assays. Intercalating dyes are generally aromatic cations with planar structures that insert between stacked base pairs in the product duplex, an arrangement that provides an environmentally dependent fluorescence enhancement for dye molecules and creates a large increase in fluorescence signal relative to the dye free solution. The assay can be utilized in low-source centers and field testing such as conferences, sports meetings, refugee camps, companies, and schools.

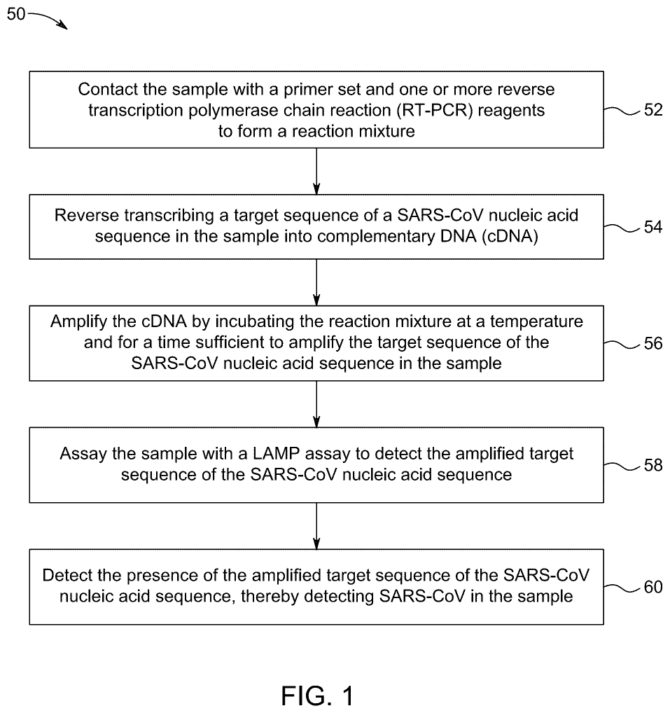

FIG. 1 illustrates a flow chart of method 50 of detecting SARS-CoV-2 in a sample. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes contacting the sample with a primer set and one or more reverse transcription loop-mediated isothermal amplification (RT-LAMP) reagents to form a reaction mixture. The sample includes a body fluid such as blood, saliva, sweat, urine, lymph, feces, and the like, or a nasal swab, an oral swab obtained from a subject. In some examples, the subject is a mammal. In one example, the mammal is a human. In some embodiments, the sample is a purified nucleic acid, a nucleic acid in a biological fluid, or a nucleic acid preparation in the swab or body fluid. In some embodiments, the method includes extracting nucleic acid from the sample prior to contacting the sample with the primer set.

The sample is contacted with the primer set. The primer set is selected from the group consisting of N-ID5 (Set-1), E-ID1 (Set-2), RdRp-ID37 (Set-3), S-ID17 (Set-4), S-ID24 (Set-5), N-ID15 (Set-6), and N-ID15n1L (Set-7). In a specific embodiment, the primer set is S-set11 (Set-8). In an embodiment, the primer set is selected from Set-1, Set-3, Set-4, Set-5, Set-6, Set-7, and Set-8, each containing six primers, the primers having sequences of at least forward outer (F3), forward inner (FIP), loop forward (LF), backward outer (B3), backward inner (BIP), and loop backward (LB). In a preferred example of the above embodiment, Set-2 contains five primers, the primers having sequences of at least forward outer (F3), forward inner (FIP), backward outer (B3), backward inner (BIP), and loop backward (LB).

One or more primers from the primer set are contacted with the RT-LAMP reagents. In some embodiments, the RT-LAMP reagents include at least one Bst DNA polymerase, deoxyribonucleotide triphosphates (dNTPs), magnesium ions, a buffer, guanidine hydrochloride, and calcein. In a preferred embodiment, the RT-LAMP reagents include calcein.

At step 54, the method 50 optionally includes reverse transcribing a target sequence of a SARS-CoV-2 nucleic acid sequence in the sample into complementary DNA (cDNA). In some embodiments, the target sequence is deoxyribonucleic (DNA) or ribonucleic acid (RNA). In some embodiments, the target sequence of a SARS-CoV-2 nucleic acid sequence is in a viral RNA sample and the viral RNA is reverse transcribed into an amplified DNA sample. In some embodiments, the reverse transcribing may be performed as a separate and/or individual step before or during the detecting SARS-CoV-2 in a sample. In one embodiment, the reverse transcribing may be performed before contacting the sample with a primer set and one or more reverse transcription loop-mediated isothermal amplification (RT-LAMP) reagents. In some embodiments, the reverse transcribing may be performed at the same time as contacting the sample with a primer set and one or more reverse transcription loop-mediated isothermal amplification (RT-LAMP) reagents. In some embodiments, the reverse transcriptase reverse transcribes the target sequence in the sample into cDNA.

At step 56, the method 50 includes incubating the reaction mixture at a temperature and for a time sufficient to amplify a target sequence of the SARS-CoV-2 nucleic acid sequence in the sample. In some embodiments, the incubation is carried out for 20-70 minutes, preferably 25-65 minutes, preferably 30-60 minutes, preferably 35-55 minutes, preferably 40-50 minutes, or preferably up to 60 minutes. In some embodiments, the method may optionally include amplifying the cDNA by incubation.

As mentioned above, in some embodiments, reverse transcribing SARS-CoV-2 RNA and then amplifying target sequences in the cDNA of the SARS-CoV-2 nucleic acid sequence in the sample may comprise a single-step process or a two-step process. The two-step process comprises first reverse transcribing the target sequence of the SARS-CoV-2 nucleic acid sequence in the sample into complementary DNA and then amplifying target sequences within the cDNA using the primer sets described herein. In the single-step process, both the reverse transcribing of SARS-CoV-2 RNA and the amplification of target sequences in the cDNA are done at the same time or in the same mix.

At step 58, the method 50 includes assaying the sample with an assay to detect the amplified target sequence of the SARS-CoV-2 nucleic acid sequence. In some embodiments, the assay may be a SARS assay. In a preferred embodiment, the assay is a colorimetric RT-LAMP assay. The assaying includes mixing a master mix, a primer mix, the sample, calcein, and dH₂O to form a colorimetric RT-LAMP mixture and incubating the colorimetric RT-LAMP mixture in a water bath at a constant temperature lying between 40-80° C., preferably 45-75° C., preferably 50-70° C., and preferably 55-65° C. for 30-60 minutes, preferably 35-55 minutes, preferably 40-50 minutes, or preferably less than 60 minutes. In a specific embodiment, the incubating of the colorimetric RT-LAMP mixture takes place in a water bath at a constant temperature of 63° C. for up to 60 minutes.

At step 60, the method 50 includes detecting the presence of the amplified target sequence of the SARS-CoV-2 nucleic acid sequence, thereby detecting SARS-CoV-2 in the sample. In some embodiments, detecting the presence of the amplified target sequence is observed with a visual color change, a transilluminator, and an ultraviolet light. In some embodiments, an average time-to-reaction (TTR) is from 20 to 60 minutes, preferably 25-55 minutes, preferably 26-54, preferably 30-50 minutes, or preferably 35-45 minutes. In a specific embodiment, the TTR of calcein-based RT-LAMP is 40 minutes. The detecting may detect SARS-CoV-2 in the form of RNA, DNA, reverse transcribed DNA, and any other genetic material known in the art.

The primers described herein that are used to amplify target sequences in SARS-CoV-2 cDNA are preferably those described by SEQ ID Nos.: 1-6. In other embodiments, these primers may be shorter or longer than those described by SEQ ID Nos.: 1-6 as long as they are able to be used to amplify the corresponding target sequence in the SARS-CoV-2 cDNA. For example, a primer for amplification of a target sequence in SARS-CoV-2 cDNA may comprise or consist of 18-20 contiguous nucleotides of SEQ ID Nos.: 1-6 or at least 25, 30, 35, 40, 45, 50, 60, 70, 80, 90, 100, 200, 250, 300, 350, 400, 450, or 500 contiguous nucleotides comprising all or a portion of SEQ ID Nos.: 1-6 that can be used in conjunction with other PCR materials to amplify a target sequence.

In some embodiments, the primer set is N-ID5 and comprises primers SEQ ID No. 1, SEQ ID No. 2, SEQ ID No. 3, SEQ ID No. 4, SEQ ID No. 5, and SEQ ID No. 6. In an embodiment, the primer is SEQ ID No. 1 and comprises at least 10 nucleotide residues and not more than 500 nucleotide residues, preferably not more than 400 nucleotide residues, preferably not more than 300 nucleotide residues, preferably not more than 250 nucleotide residues, more preferably not more than 200 nucleotide residues, still more preferably not more than 100 nucleotide residues, still more preferably not more than 50 nucleotide residues, and still more preferably not more than 30 nucleotide residues, and contains a sequence that is at least 90%, preferably at least 92%, preferably at least 95%, and more preferably at least 97% identical to SEQ ID No. 1.

In an embodiment, the primer is SEQ ID No. 2 and comprises at least 10 nucleotide residues and not more than 500 nucleotide residues, more preferably not more than 200 nucleotide residues, still more preferably not more than 100 nucleotide residues, still more preferably not more than 50 nucleotide residues, and still more preferably not more than 30 nucleotide residues, and contains a sequence that is at least 90%, preferably at least 92%, preferably at least 95%, and more preferably at least 97% identical to SEQ ID No. 2.

In an embodiment, the primer is SEQ ID No. 3 and comprises at least 10 nucleotide residues and not more than 500 nucleotide residues, more preferably not more than 200 nucleotide residues, still more preferably not more than 100 nucleotide residues, still more preferably not more than 50 nucleotide residues, and contains a sequence that is at least 90%, preferably at least 92%, preferably at least 95%, and more preferably at least 97% identical to SEQ ID No. 3.

In an embodiment, the primer is SEQ ID No. 4 and comprises at least 10 nucleotide residues and not more than 500 nucleotide residues, more preferably not more than 200 nucleotide residues, still more preferably not more than 100 nucleotide residues, still more preferably not more than 50 nucleotide residues, and contains a sequence that is at least 90%, preferably at least 92%, preferably at least 95%, and more preferably at least 97% identical to SEQ ID No. 4.

In an embodiment, the primer is SEQ ID No. 5 and comprises at least 10 nucleotide residues and not more than 500 nucleotide residues, more preferably not more than 200 nucleotide residues, still more preferably not more than 100 nucleotide residues, still more preferably not more than 50 nucleotide residues, and still more preferably not more than 30 nucleotide residues, and contains a sequence that is at least 90%, preferably at least 92%, preferably at least 95%, and more preferably at least 97% identical to SEQ ID No. 5.

In an embodiment, the primer is SEQ ID No. 6 and comprises at least 10 nucleotide residues and not more than 500 nucleotide residues, more preferably not more than 200 nucleotide residues, still more preferably not more than 100 nucleotide residues, still more preferably not more than 50 nucleotide residues, and still more preferably not more than 30 nucleotide residues, and contains a sequence that is at least 90%, preferably at least 92%, preferably at least 95%, and more preferably at least 97% identical to SEQ ID No. 6.

In some embodiments, the primer set is N-ID5, and the detection of the presence of the amplification product for the target sequence of a SARS-CoV-2 nucleic acid sequence includes an accuracy of at least 94%, preferably 95 to 100%, preferably 96 to 99%, or preferably 97 to 98%, for the colorimetric RT-LAMP assay. In a specific embodiment, the primer set is N-ID5, and the detection of the presence of the amplification product for the target sequence of a SARS-CoV-2 nucleic acid sequence includes an accuracy of 94.03%. The accuracy is based on colorimetric RT-LAMP assay results compared to reverse transcription real-time polymerase chain reaction (RT-qPCR) assay results of the sample with a sample size of at least 65 samples, preferably 66 to 70 samples, preferably 67 to 69 samples, preferably 68 samples. In a specific embodiment, the accuracy is based on colorimetric RT-LAMP assay results compared RT-qPCR assay results of the sample with a sample size of 67 samples.

In some embodiments, the primer set is N-ID5, and the assay has a specificity of at least 99%, preferably 99.1 to 100%, preferably 99.2 to 99.9%, preferably 99.3 to 99.8%, preferably 99.4 to 99.7%, or preferably 99.5 to 99.6%, for the colorimetric RT-LAMP assay. In a specific embodiment, the primer set is N-ID5, and the assay has a specificity of 100%, for the colorimetric RT-LAMP assay. The specificity is based on colorimetric RT-LAMP assay results compared to RT-qPCR assay results of the sample with a sample size of at least 65 samples, preferably 66 to 70 samples, preferably 67 to 69 samples, or preferably 68 samples. In a preferred embodiment, the specificity is based on colorimetric RT-LAMP assay results compared to RT-qPCR assay results of the sample with a sample size of 67 samples.

In some embodiments, the primer set is N-ID5, and the assay has a sensitivity of at least 90%, preferably 91 to 100%, preferably 92 to 99%, preferably 93 to 98%, preferably 94 to 97%, or preferably 95 to 96%, for the colorimetric RT-LAMP assay. In a specific embodiment, the primer set is N-ID5, and the assay has a sensitivity of 90.91% for the colorimetric RT-LAMP assay. The sensitivity is based on colorimetric RT-LAMP assay results compared to RT-qPCR assay results of the sample with a sample size of at least 65 samples, preferably 66 to 70 samples, preferably 67 to 69 samples, or preferably 68 samples. In a preferred embodiment, the sensitivity is based on colorimetric RT-LAMP assay results compared to RT-qPCR assay results of the sample with a sample size of 67 samples.

In some embodiments, the primer set is N-ID5, and a positive-percent agreement (PPA) of the RT-LAMP assay of the sample is at least 99%, preferably 99.1 to 100%, preferably 99.3 to 99.8%, or preferably 99.5 to 99.7%, based on a true positive and a false positive when compared to real-time reverse-transcriptase polymerase chain reaction (RT-qPCR) assay results of the sample with a sample size of at least 65 samples, preferably 66 to 70 sample, preferably 67 to 69 samples, or preferably 68 samples. In a specific embodiment, the primer set is N-ID5, and the PPA of the RT-LAMP assay of the sample is 100%, based on a true positive and a false positive when compared to RT-qPCR assay results of the sample with a sample size of 67 samples.

In some embodiments, the primer set is N-ID5 and a negative-percent agreement (NPA) of the RT-LAMP assay of the sample is at least 85%, preferably 85 to 100%, preferably 87 to 99%, preferably 88 to 98%, preferably 89 to 97%, preferably 90 to 96%, preferably 91 to 95%, or preferably 92 to 94%, based on a true negative and a false negative when compared to RT-qPCR assay results of the sample with a sample size of at least 65 samples preferably 66 to 70 samples, preferably 67 to 69 samples, or preferably 68 samples. In a specific embodiment, the primer set is N-ID5 and the NPA of the RT-LAMP assay of the sample is 85.19%, based on a true negative and a false negative when compared to RT-qPCR assay results of the sample with a sample size of 67 samples.

The method of the present disclosure embodies the salient features of detecting the variants of the SARS-CoV-2 virus. Thus, in an exemplary embodiment, the SARS-CoV-2 is selected from the group consisting of SARS-CoV-2, SARS-CoV-HKU-39849 (e.g., AY278491.2), SARS-CoV-TW11 (e.g., AY502924.1), SARS-CoV-TW4 (e.g., AY502927.1), SARS-CoV-Sin846 (e.g., AY559094.1), SARS-CoV-GZ0402 (e.g., AY613947.1), and SARS-CoV-Tor2 (e.g., NC_004718.3). In one embodiment, the method and kit of the present disclosure can detect a mixture of SARS-CoV-2 variants in the sample. In an embodiment, the method and kit of the present disclosure may detect any SARS-CoV-2 variants, known and unknown in the art, in the sample.

In some embodiments, the SARS-CoV-2 is SARS-CoV-2. The SARS-CoV-2 includes at least one variant selected from the following group consisting of alpha (B.1.1.7), beta (B.1.351), gamma (P.1), delta (B.1.617.2), and omicron (B.1.1.529). In some examples, the SARS-CoV-2 is detected among other viruses, including parainfluenza 3, enterovirus, rhinovirus, human metapneumovirus A+B, and parainfluenza virus 4, bocavirus, or coronavirus 229 E.

The detection of SARS-CoV-2 is a challenge for public health. Rapid and accurate detection is needed, especially during a pandemic, as seen recently worldwide. In such a scenario, the method of detection plays a role in terms of clinical and epidemiological impact. One of the main challenges is identifying the infection at lower concentrations. The limit of detection (LoD) refers to the lowest detectable concentration of viral RNA that returns a positive result in >95% of repeat measurements. A high LoD uses more copies of viral RNA per sample, leading to a high proportion of false negatives for those who are at the beginning of an infection or are asymptomatic. Therefore, LoD is a feature that determines the quality of a method for detecting the SARS-CoV-2 in a sample. In some embodiments, when the primer set is N-ID5, a limit of detection (LoD) of the assay is 0.01-0.2 copies/μL of the sample, preferably 0.02-0.19 copies/μL of the sample, preferably 0.03-0.18 copies/μL of the sample, preferably 0.04-0.17 copies/μL of the sample, preferably 0.05-0.16 copies/μL of the sample, preferably 0.06-0.15 copies/μL of the sample, preferably 0.07-0.14 copies/μL of the sample, preferably 0.08-0.13 copies/μL of the sample, preferably 0.09-0.12 copies/μL of the sample, and more preferably 0.10-0.11 copies/μL of the sample. In a specific embodiment, when the primer set is N-ID5, the LoD of the assay is 0.1 copies/μL of the sample.

In some embodiments, the target sequence of the SARS-CoV-2 nucleic acid sequence is located within at least one gene selected from the group consisting of nucleocapsid (N), spike (S), RNA-dependent RNA polymerase (RdRp), and envelope (E) of a SARS-CoV-2 genome.

In some embodiments, the primer sets N-ID5 (Set-1), E-ID1 (Set-2), RdRp-ID37 (Set-3), S-ID17 (Set-4), S-ID24 (Set-5), N-ID15 (Set-6), N-ID15n1L (Set-7), and S-set11 (Set-8) may be used in any PCR, qPCR, RT-PCR, RT-qPCR, LAMP, RT-LAMP, and any other PCR-based methods to detect SARS-CoV-2 in a sample.

In some embodiments, the method further includes treating a subject for which the sample was obtained. In some embodiments, treating a subject may include a course of one or more antiviral drugs, a course of one or more antiviral agents, one or more vaccines, and any other treatment processes known in the art.

In another exemplary embodiment, a kit for detection of SARS-CoV-2 in a sample is disclosed. Kits refer to a combination of material that are needed to perform a reaction. The kit includes a reverse transcriptase, a polymerase, calcein, and a universal primer set for loop-mediated isothermal amplification (LAMP) of a target sequence in a SARS-CoV-2 nucleic acid sequence and variants thereof containing mutations within one or more primer binding sites. The universal primer set suitable for LAMP is capable of hybridizing to the target sequence of a SARS-CoV-2 nucleic acid sequence in the presence of a plurality of undefined mutations and is configured to provide a positive result for the target sequence of a SARS-CoV-2 nucleic acid sequence in a predetermined assay time period otherwise determined for a positive sample of a target nucleic acid having a known sequence. Any of the reagents in the kit may be combined in a mixture in a single container or provided in separate containers. In some embodiments, the kit may include a mixture of lyophilized reagents and reagents in a storage buffer. In one embodiment, the kit may contain multiple tubes with the master mix in a first tube, the primer mix in a second tubes, and, optionally, other reagents in different tubes. In one example, the kit may analyze a plurality of different target nucleic acids. In addition, the kit may also include a printed and electronic user's guide describing the steps for performing the detection assay.

Accordingly, an aspect of the present disclosure is to provide primers and a method of detection of viruses of interest, including SARS-CoV-2. The kit of the present disclosure exhibited the advantage of the ready-to-use reagents to meet any emergency, such as the COVID-19 pandemic. Yet another advantage of the kit is that the kit can detect all the known variants of the SARS-CoV-2 virus.

### EXAMPLES

The disclosure will now be illustrated with working examples intended to demonstrate the working of the disclosure and not to restrictively imply any limitations on the scope of the present disclosure. The working examples depict an example of the method of the present disclosure.

The following examples demonstrate the method of detecting SARS-CoV-2 in the sample. The examples are provided solely for illustration and are not to be construed as limitations of the present disclosure, as many variations thereof are possible without departing from the spirit and scope of the present disclosure.

### Example 1: SARS-CoV-2 Genome Sequences and Primer Design

SARS-CoV-2 genomes belong to the variant of interest (VOI) and the variant of concern (VOC) including B.1.1.7 (alpha), B.1.351 (Beta), P.1 (Gamma), B.1.617.2 (Delta), and B.1.1.529 (Omicron) which was downloaded from GISAID database, gisaid.org. The sequences originating from different countries and continents were collected. A multiple alignment using fast fourier transform (MAFFT) software, mafft.cbrc.jp, was used to align genomes with default settings. The gene sequences (N, RdRp, E, and S) were selected and screened by using JalView (v2.11.1.3) program to check possible mutation sites. Only the consensus sequences (100% alignments) were chosen to design the primers. Primer Explorer V5 by Eiken (primerexplorer.jp) was used for primer design. Several LAMP primer sets were designed to acquire the best sensitivity and specificity [Alhamid, G., Tombuloglu, H., & Al-Suhaimi, E. (2023). Development of loop-mediated isothermal amplification (LAMP) assays using five primers reduces the false-positive rate in COVID-19 diagnosis. Scientific Reports, 13(1), 1-13, incorporated herein by reference in its entirety]. Possible secondary structures like homodimer, heterodimer, and hairpins were inspected using the Integrated DNA Technologies (IDT) DNA OligoAnalyzer tool, idtdna.com.

### Example 2: Sample Collection and RNA Isolation

The RNA samples were extracted from nasopharyngeal swabs or combined nasopharyngeal/oral swabs collected from patients at King Fahad Specialist Hospital (KFSH) and John Hopkins Aramco Healthcare (JHAH), Dammam/KSA. The swabs were placed in a liquid viral transport medium (VTM) from Capricorn Scientific (Germany). The collected samples were brought to the diagnostic laboratory in sterile containers to either proceed directly or keep at +4° C. for further processing. RNA was isolated using a QIAamp Viral RNA kit (Qiagen, Germany) in accordance with the manufacturer's instructions, quantified by using NanoDrop 2000 (Thermo Sci.), and stored at −80° C. for further experiments.

### Example 3: RT-LAMP Conditions

All master-mix preparations for the RT-LAMP reaction were performed on ice inside a biosafety cabinet level-2 (BSL-2). RT-LAMP reactions were performed according to New England BioLabs (NEB) recommendations, containing the following components: 2.5 μL 10× Isothermal Amplification Buffer II, 2.5 μL MgSO₄ (100 mM), 3.5 μL dNTP (10 mM), 0.21 μL Bst 3.0 DNA Polymerase (120,000 U), 2.5 μL 10× primers mix (FIP and BIP (1.6 M), F3 and B3 (0.2 M), LF and/or LB (0.4 M)), 1 μL guanidine hydrochloride (40 mM), 1 μL fluorescent calcein (2.5 mM), and 2 μL template RNA. Ultra-pure DNase/RNase-free distilled water was added in quantity enough to complete the final volume reaction of 25 μL. Isothermal amplification was performed on either a TECHNE thermal cycler or a water bath at 63° C. constant temperature for up to 60 minutes. The products were monitored at 10-minute intervals and stored at 4° C.

### Example 4: Detection of SARS-CoV-2

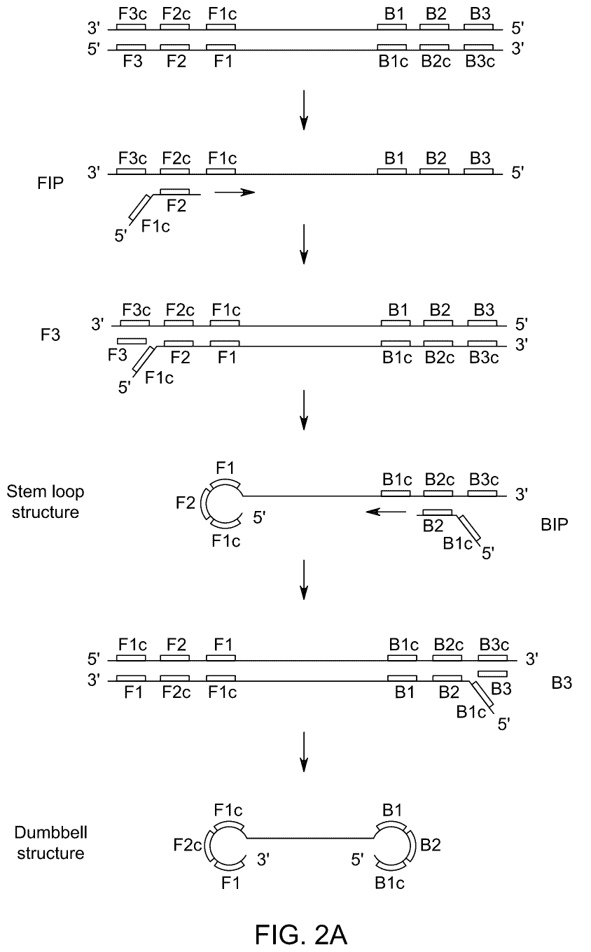

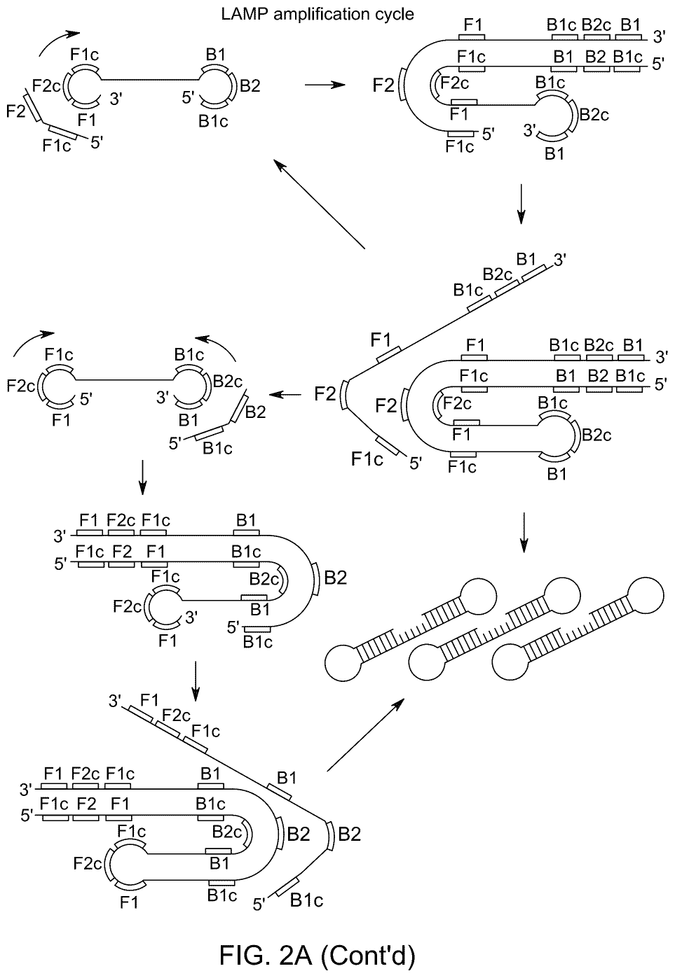

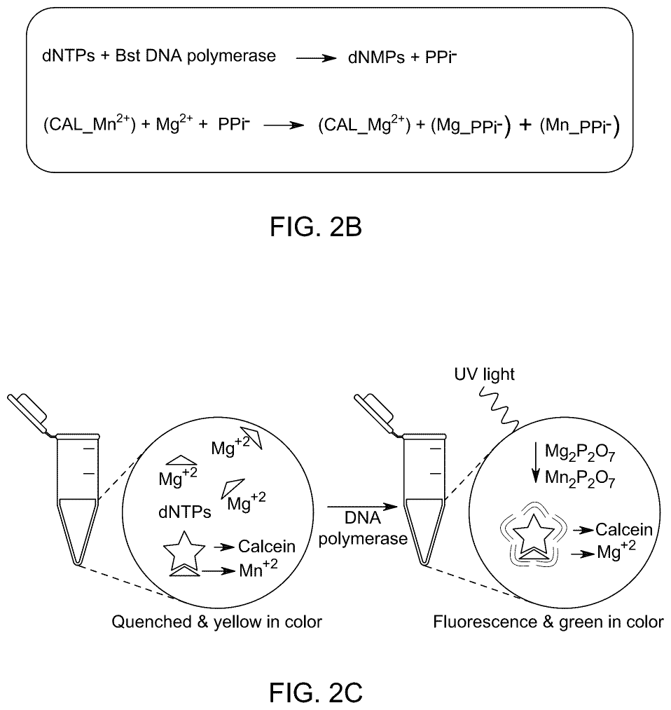

Calcein, a metal indicator dye, allows visual detection of the reactant color change from orange to green based on substituting manganese chloride (MnCl₂) to magnesium chloride (MgCl₂). FIG. 2A depicts the binding position of six loop-mediated isothermal amplification (LAMP) primers, namely LB, LF, FIP, BIP, F3, and B3, and the formation of stem-loop and dumbbell structures, which serve as starting points for the LAMP amplification cycles; and FIGS. 2B-2C depict the input and output of the LAMP reaction upon the activation of the Bst DNA polymerase. The reaction was considered positive when the naked eye observed orange and/or yellow to green color change. In addition, the products were visualized both under a transilluminator and mini hand-UV with UV-protective glasses (FIG. 2B). In addition, RT-LAMP products were loaded on 2% agarose gel and run in a gel electrophoresis device for 40 minutes at 100 V constant voltage.

### Example 5: Amplification Efficiency and Analytical Sensitivity

The assay's performance was assessed regarding limit-of-detection (LoD) using 10-fold diluted samples of SARS-CoV-2 positive control (PC) RNA from 10⁰ to 10⁶. Also, the effect of enzyme concentration in color development and the detection of SARS-CoV-2 were tested using different Bst3.0 enzyme concentrations: 1×, 2×, and 3×, corresponding to 8,000, 16,000, and 24,000 U/mL, respectively. The analytical specificity of the primers used for RT-LAMP was determined using the RNA specimens isolated from other respiratory viruses, including parainfluenza virus 3, enterovirus, rhinovirus, human metapneumovirus A+B, parainfluenza virus 4, bocavirus, and coronavirus 229 E.

### Example 6: Diagnostic Performance

Clinical RNA samples (n=67) were tested using calcein-based RT-LAMP, multiplex RT-PCR targeting viral RdRp and N genes, and human RP genes [Tombuloglu, H., Sabit, H., Al-Khallaf, H., Kabanja, J. H., Alsaeed, M., Al-Saleh, N., & Al-Suhaimi, E. (2022). Multiplex real-time RT-PCR method for the diagnosis of SARS-CoV-2 by targeting viral N, RdRP and human RP genes. Scientific Reports, 12(1), 2853; and Tombuloglu, H., Sabit, H., Al-Suhaimi, E., Al Jindan, R., & Alkharsah, K. R. (2021). Development of multiplex real-time RT-PCR assay for the detection of SARS-CoV-2. Plos one, 16(4), e0250942, both of which are incorporated herein by reference in its entirety]. An RNA mixture containing synthetic copies of each target gene (Twist synthetic RNA control 51 (EPI_ISL_7718520), Twist Bioscience, USA) was used as the positive control (PC) sample. For negative control (NC), RNase/DNase-free dH₂O was added instead of the RNA template. Reaction mixtures were segregated using 2% agarose gel electrophoresis stained with VisualaNA(A) DNA to confirm the LAMP results. Gels were imaged under UV irradiation with a transilluminator from Bio-Rad Laboratories. The sensitivity, specificity, accuracy, positive-percent agreement (PPA), and negative-percent agreement (NPA) were calculated according to the following equations:

$$\begin{matrix}Sensitivity = \frac{T P}{T P + F N} \\ Specificity = \frac{T N}{T N + F P} \\ Accuracy = \frac{T P + T N}{T P + T N + F P + F N} \\ NPA = \frac{T N}{T N + F N} \\ PPA = \frac{T P}{T P + F P}\end{matrix}$$

where TP is true positive, FN is false negative, FP is false positive, and TN is true negative. The specific sequences of the primers used are provided in Table 2 below.

TABLE 2
Sequences of each RT-LAMP primer.

| Primer | SEQ. | |
|---|---|---|
| set | ID No. | Sequence (5′ - 3′) |
| N-ID5 | 1 | F3: CCAGAATGGAGAACGCAGTG |
| (Set-1) | 2 | B3: CCGTCACCACCACGAATT |
| | 3 | FIP: AGCGGTGAACCAAGACGCAGGGCGCGATCAAAACAACG |
| | 4 | BIP: |
| | | AATTCCCTCGAGGACAAGGCGAGCTCTTCGGTAGTAGCCAA |
| | 5 | LF: TTATTGGGTAAACCTTGGGGC |
| | 6 | LB: TCCAATTAACACCAATAGCAGTCCA |
| E-ID1 | 7 | F3: TCATTCGTTTCGGAAGAGA |
| (Set-2) | 8 | B3: AGGAACTCTAGAAGAATTCAGAT |
| | 9 | FIP: |
| | | TGTAACTAGCAAGAATACCACGAAACAGGTACGTTAATAGTT |
| | | AATAGCG |
| | 10 | BIP: |
| | | GCTTCGATTGTGTGCGTACTCGAGAGTAAACGTAAAAAGAAG |
| | | G |
| | 11 | LB: GCTGCAATATTGTTAACGTGAGTC |
| RdRp- | 12 | F3: ACAAAGCCTTACATTAAGTGG |
| ID37 | 13 | B3: CACCATCAACAAATATTTTTCTCAC |
| (Set-3) | 14 | FIP: |
| | | TGGGTGGTATGTCTGATCCCAATAGATTTGTTAAAATATGACT |
| | | TCACGG |
| | 15 | BIP: |
| | | TGTGTTAACTGTTTGGATGACAGATTGTAAGTGGGAACACTGT |
| | 16 | LF: ACGGTCAAAGAGTTTTAACCTCTCT |
| | 17 | LB: GCATTCTGCATTGTGCAAACT |
| S-ID17 | 18 | F3: TCTTTCACACGTGGTGTT |
| (Set-4) | 19 | B3: GTACCAAAAATCCAGCCTC |
| | 20 | FIP: |
| | | CATGGAACCAAGTAACATTGGAAAACCTGACAAAGTTTTCAG |
| | | ATCC |
| | 21 | BIP: |
| | | CTCTGGGACCAATGGTACTAAGAGGACTTCTCAGTGGAAGCA |
| | 22 | LF: GGTAAGAACAAGTCCTGAGTTGAA |
| | 23 | LB: GTTTGATAACCCTGTCCTACCATT |
| S-ID24 | 24 | F3: GGTGTTTATTACCCTGACAAAG |
| (Set-5) | 25 | B3: GTACCAAAAATCCAGCCTC |
| | 26 | FIP: |
| | | CATGGAACCAAGTAACATTGGAAAATTTTCAGATCCTCAGTTT |
| | | TACATTC |
| | 27 | BIP: |
| | | CTCTGGGACCAATGGTACTAAGAGGACTTCTCAGTGGAAGCA |
| | 28 | LF: GAAAGGTAAGAACAAGTCCTGAGT |
| | 29 | LB: GTTTGATAACCCTGTCCTACCATT |
| N-ID15 | 30 | F3: AGATCACATTGGCACCCG |
| (Set-6) | 31 | B3: CCATTGCCAGCCATTCTAGC |
| | 32 | FIP: |
| | | TGCTCCCTTCTGCGTAGAAGCCAATGCTGCAATCGTGCTAC |
| | 33 | BIP: GGCGGCAGTCAAGCCTCTTCCCTACTGCTGCCTGGAGTT |
| | 34 | LF: GGCAATGTTGTTCCTTGAGGAAGTT |
| | 35 | LB: CACGTAGTCGCAACAGTTCAA |
| N- | 36 | F3: AGATCACATTGGCACCCG |
| ID15n1L | 37 | B3: CCATTGCCAGCCATTCTAGC |
| (Set-7) | 38 | FIP: |
| | | TGCTCCCTTCTGCGTAGAAGCCAATGCTGCAATCGTGCTAC |
| | 39 | BIP: GGCGGCAGTCAAGCCTCTTCCCTACTGCTGCCTGGAGTT |
| | 40 | LF: GCAATGTTGTTCCTTGAGGAAGTT |
| | 41 | LB: GTTCCTCATCACGTAGTCGCAACA |
| S-set11 | 42 | F3: TCTATTGCCATACCCACAA |
| (Set-8) | 43 | B3: GTACCAAAAATCCAGCCTC |
| | 44 | FIP: |
| | | CATGGAACCAAGTAACATTGGAAAATTTTCAGATCCTCAGTTT |
| | | TACATTC |
| | 45 | BIP: |
| | | CTCTGGGACCAATGGTACTAAGAGGACTTCTCAGTGGAAGCA |
| | 46 | LF: GAAAGGTAAGAACAAGTCCTGAGT |
| | 47 | LB: GTTTGATAACCCTGTCCTACCATT |

Abbreviations: F3, forward outer; FIP, forward inner; LF, loop forward; B3, backward outer; BIP, backward inner; LB, loop backward. The primers were designed by using the PrimerExplorer program.

### Observation of the LAMP Reaction

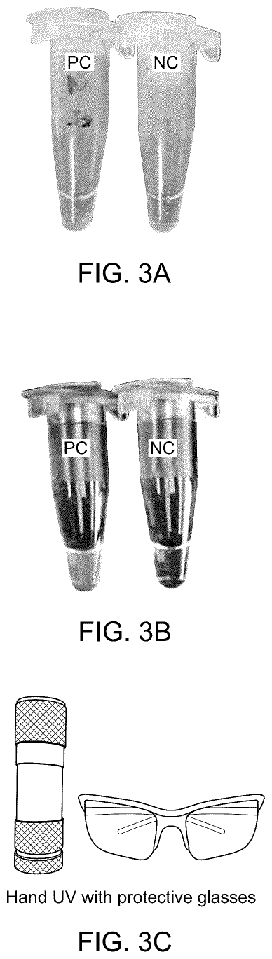

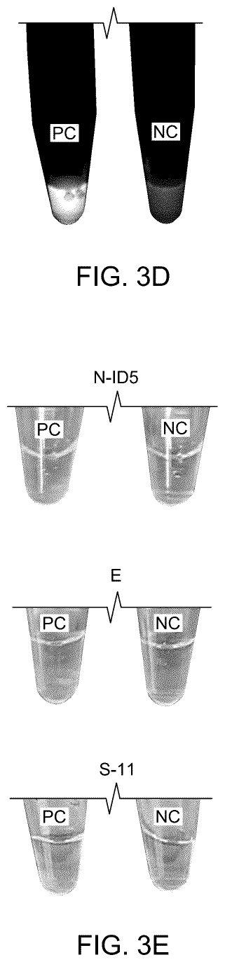

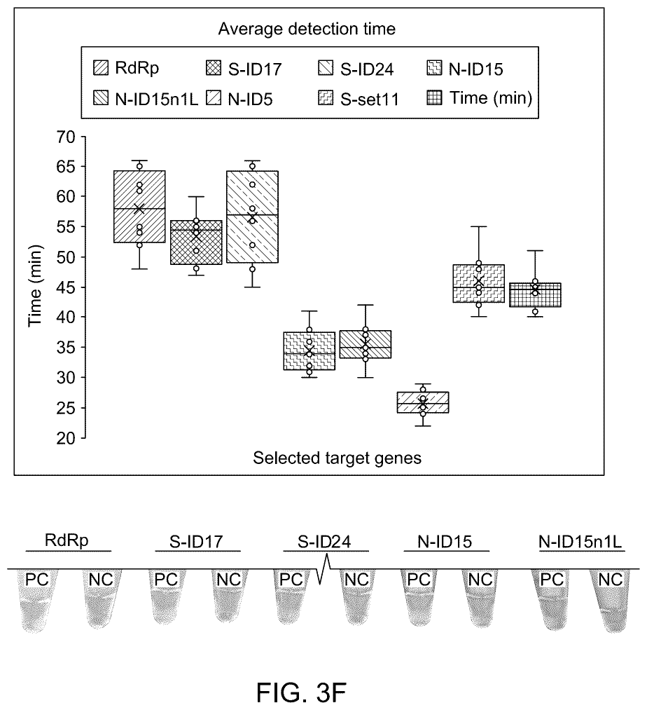

LAMP is a well-known diagnostic technique for screening infections in routine or field station laboratories. By integrating the LAMP technique with dye indicators, colorimetric LAMP can be produced for quick and accurate pathogen identification. In this study, a single-tube colorimetric LAMP assay was demonstrated and optimized for visual detection of the SARS-CoV-2 virus using calcein dye, resulting in a clear distinction between the reactions diagnosed as positive or negative. The metal indicator dye, calcein, allows visual observation by the naked eye or under UV light. A color change to green was observed in PC samples after colorimetric RT-LAMP reactions, while the non-template or negative control (NC) was orange (FIG. 3A). Under UV light, the color turns light green from dark green (FIG. 3B). However, a hand UV lamp with UV-protective glass must be utilized (FIG. 3C). In addition, the color change can be observed under a transilluminator, as depicted in FIG. 3D.

Detection of SARS-CoV-2 with Calcein by Using Different Primer Sets

Different LAMP primer sets (n=8) targeting four other genes (N, S, RdRp, and E), namely N-ID5, E-ID1, S-set11, RdRp-ID37, S-ID17, S-ID24, N-ID15, and N-ID15n1L, were designed to find out the efficient primer set for the best result (FIG. 3E). The reactions took place in a thermal block or a water bath. The average detection time of reaction mixtures, including different primers, ranges between 25 to 60 minutes, as shown in FIG. 3F. The mean detection time (n=6) was about 26.2 minutes when using the N-ID5 set, followed by N-ID15n1L (34.4 minutes), N-ID15 (35.2 minutes), E-ID1 (44.8 minutes), and S-set11 (45.1 minutes). The mean detection time for the S-ID17, S-ID24, and RdRp-ID37 primer sets was relatively late (˜55-57 minutes). Accordingly, N-ID5 was determined as the primer set, giving the earliest result and used in further steps.

### Effect of Enzyme Concentration on Color Development

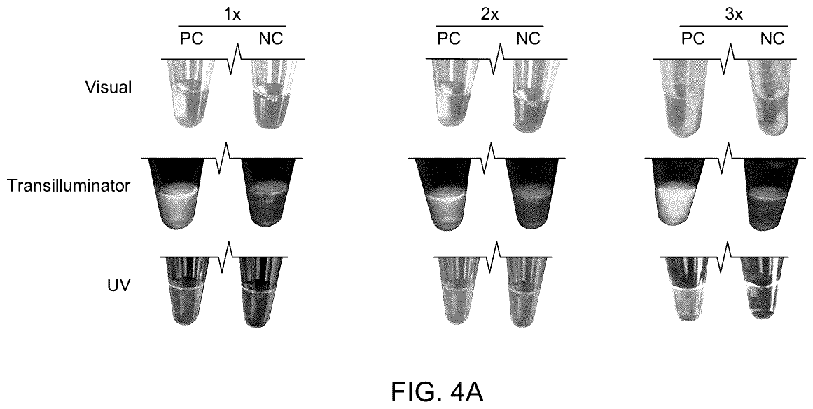

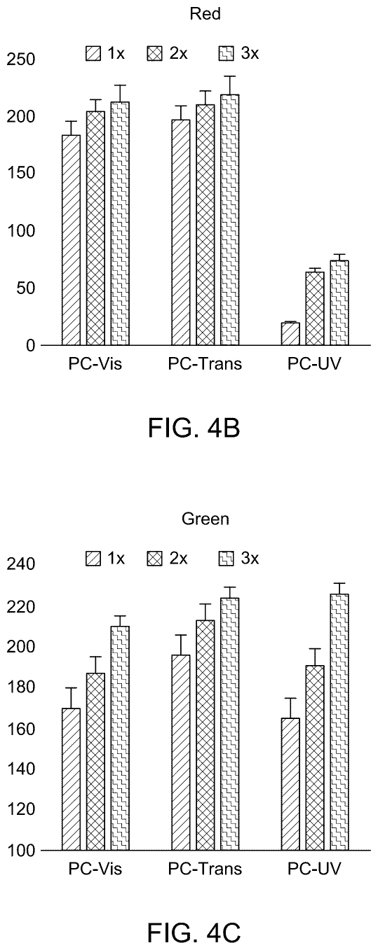

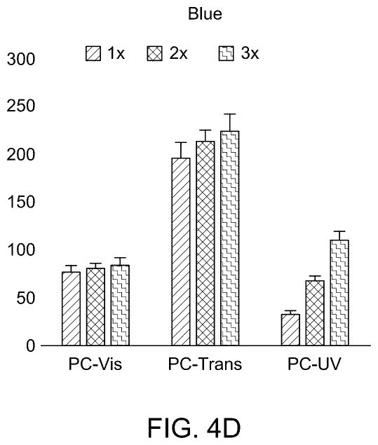

To elucidate the effect of enzyme concentration on color development and intensity, three different Bst 3.0 enzyme concentrations (8,000 U/mL, 16,000 U/mL, and 24,000 U/mL, corresponding to 1×, 2×, and 3×, respectively) were used with N-ID5 primer set. When examined under a transilluminator, UV light, or by the naked eye, the triplicate concentration (24,000 U/ml) led to the highest color intensity among other engagements (FIG. 4A). The changes in the color intensity were also evident by reading the red, green, and blue (RGB) codes using a mobile application, as depicted in FIGS. 4B-4D. It is evident that increasing the enzyme concentration gradually raised the RGB value and color intensity in all tested observation assays (i.e., visual (Vis), transilluminator (Trans), and UV light (UV)). Compared to the visual (Vis) and transilluminator (Trans) tests, the UV light revealed the most notable RGB difference between the PC specimens at 1× and 3× enzyme concentrations.

### Detection of SARS-CoV-2 on Clinical Samples

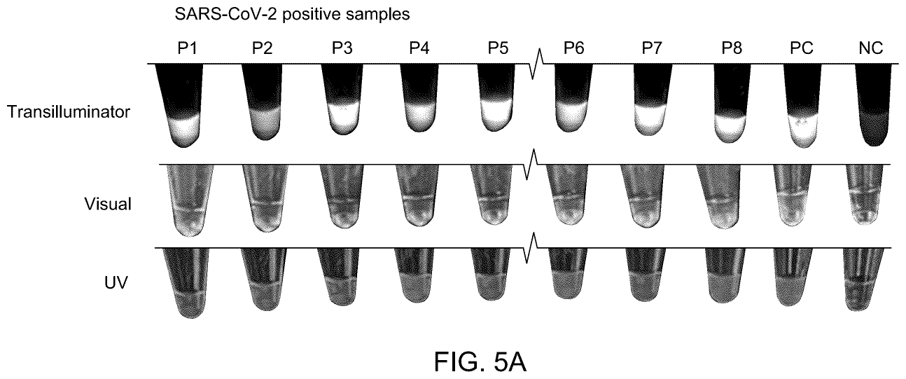

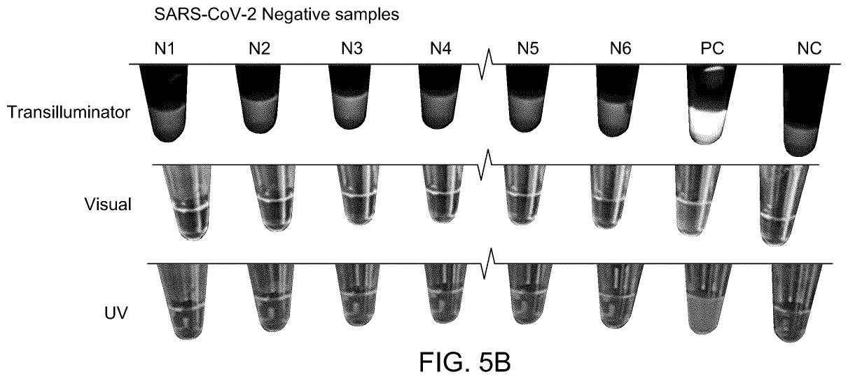

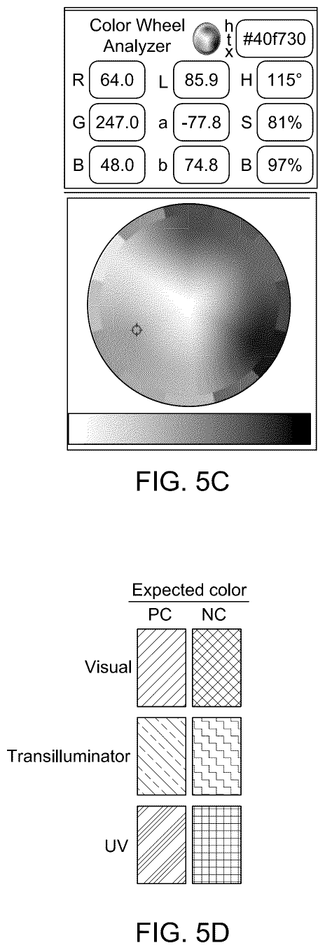

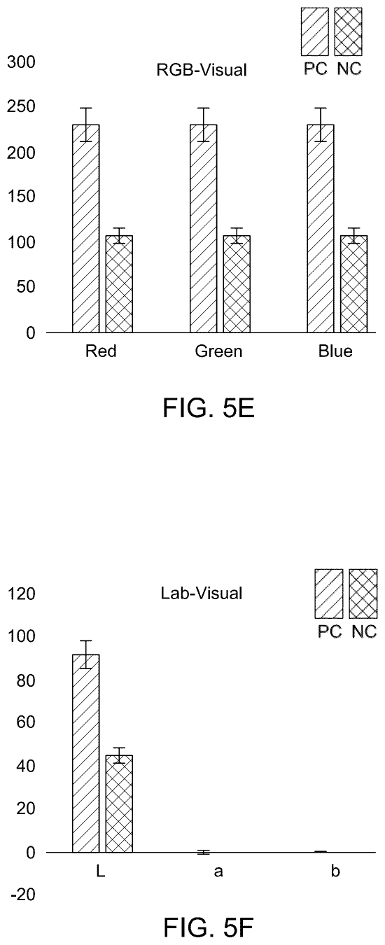

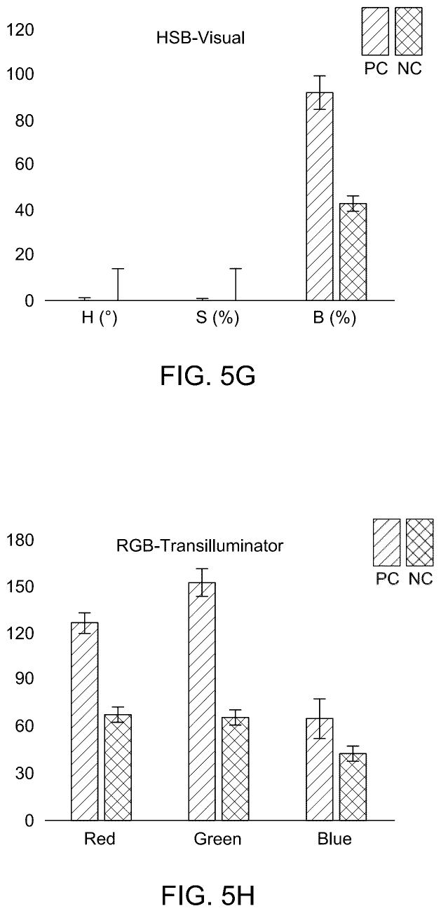

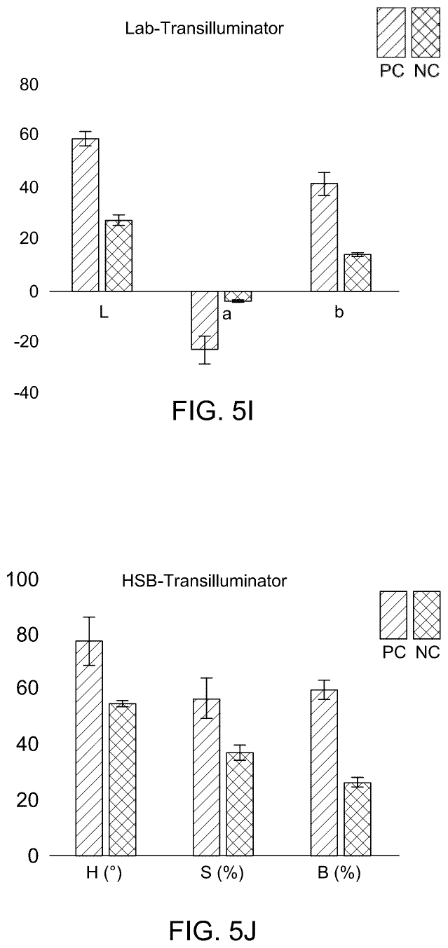

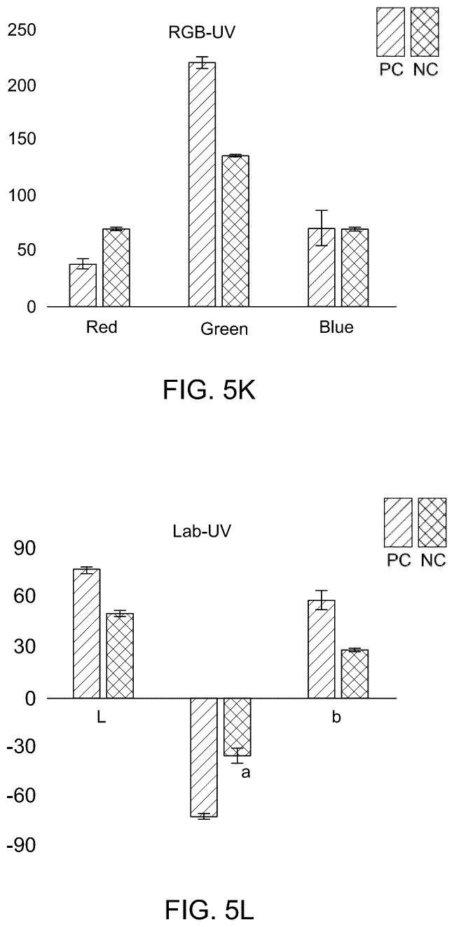

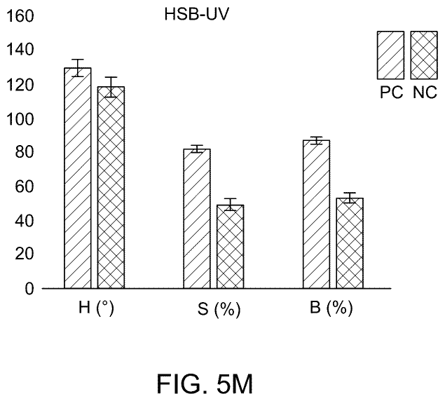

The color change after the calcein-LAMP reaction was observed by using clinically verified SARS-CoV-2 positive (n=8) (FIG. 5A) and negative (n=6) (FIG. 5B) samples. The color difference between the positive and negative tubes was evident in all observation assays, i.e., transilluminator, UV light, and naked eye (visual). Together with this, the color change was quantified by using a mobile color analyzer application (Color Wheel Analyzer, CWA), which numerically defines the RGB, Lab, and HSB value on a picture in either .jpeg or .png format (FIG. 5C). The colors in SARS-CoV-2 positive and negative samples upon the calcein-LAMP reaction were indicated in FIG. 5D. The results in visual observation showed that the Red, Green, Blue, L, and B values quite different between the SARS-CoV-2 negative and positive samples, but no results were obtained for the a, b, H, and S values (FIGS. 5E-5G). Under the transilluminator, the R, G, L, a, b, and B values were altered between the SARS-CoV-2 negative and positive reaction tubes (FIGS. 5H-5J). Under UV light, the CWA program resulted in the most color difference in Green (RGB), a (Lab), and S (HSB) values (FIGS. 5K-5M). In addition to the naked eye option, the users can choose one or more of these color code models to identify positive and negative samples according to the existence of these facilities (i.e., UV, transilluminator, or visual).

### Diagnostic Performance of Calcein-LAMP on Clinical Samples

Clinical samples (n=67) were comparatively tested by RT-LAMP assay using an N-ID5 primer set and optimized LAMP protocol. Multiplex RT-qPCR assays targeting viral N and RdRp, and human RP genes were used to verify the SARS-CoV-2 positive and negative samples. Among the tested samples, the calcein-LAMP assay was compatible with RT-qPCR results in 63 samples (94.03%), as 40 were detected as SARS-CoV-2 positives and 23 were negatives in both assays, as shown in Table 1. The sensitivity (90.91%), accuracy (94.03%), specificity (100%), positive-percent agreement (PPA) (100%), and negative-percent agreement (NPA) (85.19%) were calculated. Four samples were found inconclusive, which were positive in calcein RT-LAMP, but negative in rRT-PCR. Also, no false-negative (FN) result was evident (Table 1).

TABLE 1
Comparison of calcein RT-LAMP and rRT-PCR assays.

| | | Calcein RT-LAMP | | |
|---|---|---|---|---|
| | | (+) | (−) | Total |
| RT-qPCR | (+) | 40 (TP) | 0 (FP) | 40 (TP + FP) |
| | (−) | 4 (FN) | 23 (TN) | 27 (FN + TN) |
| Total | | 44 | 23 | 67 |

True positive (TP), false positive (FP), true negative (TN), and false negative (FN) terms were utilized to evaluate the diagnostic accuracy of the RT-LAMP method.

### Analytic Sensitivity on Different Variants: Delta and Omicron

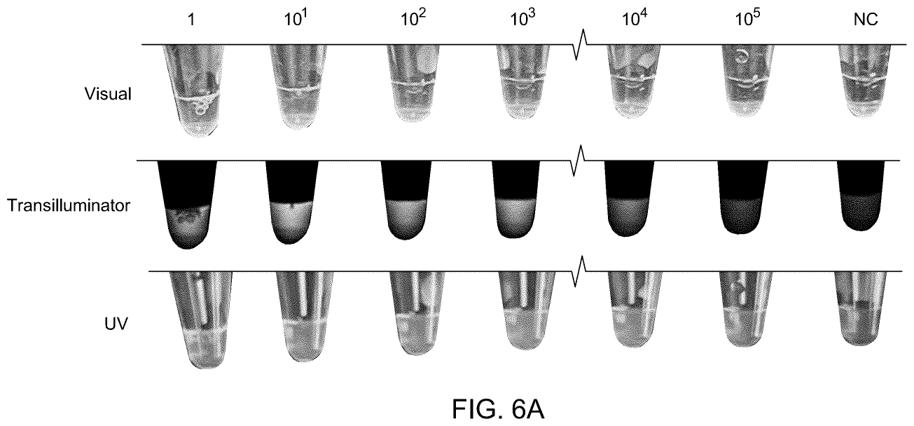

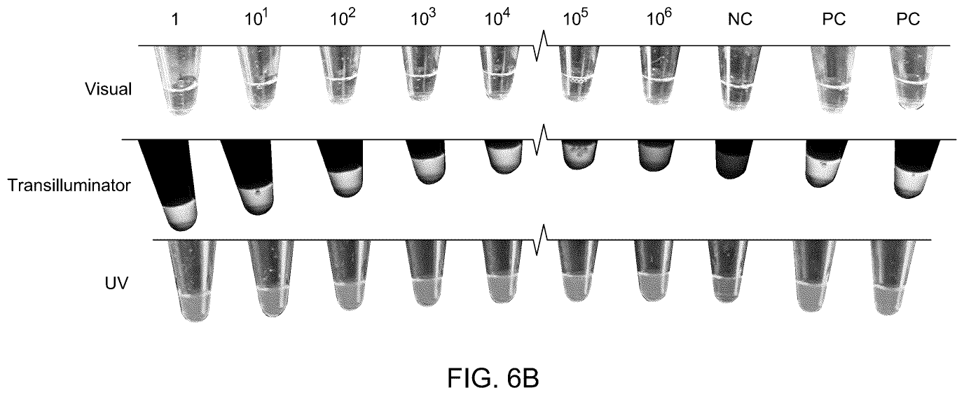

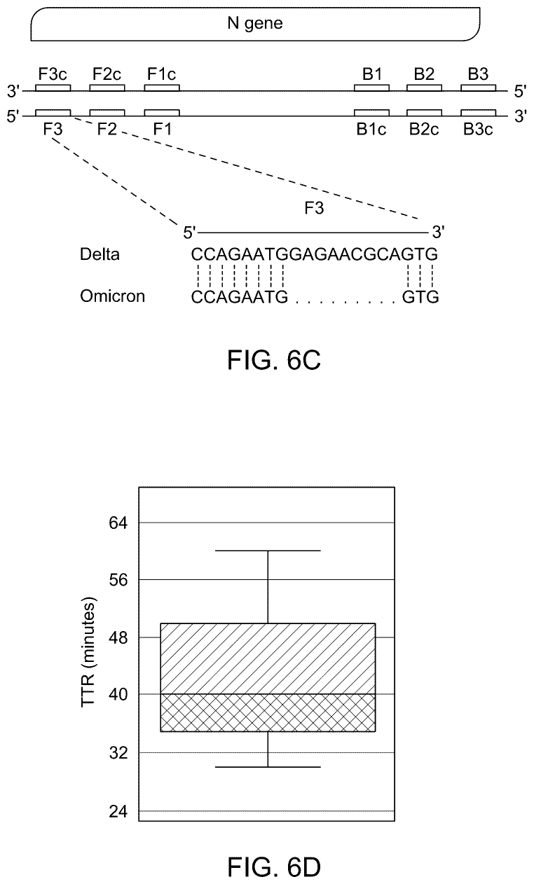

An RNA sample of SARS-CoV-2 (Delta variant) with an unknown concentration was serially diluted (1, 10¹, 10², 10³, 10⁴, and 10⁵-fold) to determine the analytical sensitivity of LAMP reactions (FIG. 6A). Results showed that color change from orange to green had been detected in 10⁰, 10¹, 10², 10³, and 10⁴-fold dilutions, while the 10⁵ and negative control (NC) samples were orange. The same result was evident under the transilluminator and UV light. Also, it was tested further via gel electrophoresis, where the results are consistent with the colorimetric analyses. A ladder-type banding pattern detected dilutions, while no bands appeared for NCs. In FIG. 6B, a known concentration of pseudo viral SARS-CoV-2 RNA (Twist Bioscience, Omicron variant) was serially diluted (1, 10¹, 10², 10³, 10⁴, 10⁵, and 10⁶-fold) and used as the template. After RT-LAMP reactions, the color change from orange to green was visually observed up to 10⁶-fold dilutions. It was also consistent with the transilluminator and UV light observations. The LoD of the assay was calculated as ˜2 copies/reaction or ˜0.1 copies/μl of the RNA sample.

When the primer binding sites were checked in the Delta and Omicron variants, it was noted that the binding position of the F3 primer included deletions (nine nucleotides) in the Omicron variant (FIG. 6C). This mutation was thought to affect the binding of the F3 primer. However, it is seen that it does not disrupt the LAMP reaction. These results showed the successful amplification of the N gene by calcein RT-LAMP regardless of variants, including Delta and Omicron. The average time-to-reaction (TTR) value of calcein-based RT-LAMP was calculated as 40 minutes (n=53) (FIG. 6D). It was previously detected that the mean TTR (n=6) was about 26.2 minutes (FIG. 3F). The average TTR may vary depending on the copy number of viral RNA in the samples.

Numerous modifications and variations of the present disclosure are possible in light of the above teachings. It is therefore to be understood that within the scope of the appended claims, the disclosure may be practiced otherwise than as specifically described herein.

1: A method of detecting SARS-CoV-2 in a sample, comprising:
contacting the sample with a primer set and one or more reverse transcription loop-mediated isothermal amplification (RT-LAMP) reagents to form a reaction mixture,
wherein the RT-LAMP reagents comprise calcein,
incubating the reaction mixture at a temperature and for a time sufficient to amplify a target sequence of the SARS-CoV-2 nucleic acid sequence in the sample;
assaying the sample with an assay to detect the amplified target sequence of the SARS-CoV-2 nucleic acid sequence; and
detecting a presence of the amplified target sequence of the SARS-CoV-2 nucleic acid sequence, thereby detecting SARS-CoV-2 in the sample,
wherein the primer set is selected from the group consisting of:
N-ID5 (Set-1),
E-ID1 (Set-2),
RdRp-ID37 (Set-3),
S-ID17 (Set-4),
S-ID24 (Set-5),
N-ID15 (Set-6), and
N-ID15n1L (Set-7).

2: A method of detecting SARS-CoV-2 in a sample, comprising:
contacting the sample with a primer set and one or more reverse transcription loop-mediated isothermal amplification (RT-LAMP) reagents to form a reaction mixture,
wherein the RT-LAMP reagents comprise calcein,
incubating the reaction mixture at a temperature and for a time sufficient to amplify a target sequence of the SARS-CoV-2 nucleic acid sequence in the sample;
assaying the sample with an assay to detect the amplified target sequence of the SARS-CoV-2 nucleic acid sequence; and
detecting a presence of the amplified target sequence of the SARS-CoV-2 nucleic acid sequence, thereby detecting SARS-CoV-2 in the sample,
wherein the primer set is S-set11 (Set-8).

3: The method of claim 1, wherein the RT-LAMP reagents comprise at least one Bst deoxyribose nucleic acid (DNA) polymerase, deoxyribonucleotide triphosphates (dNTPs), magnesium ions, a buffer, a guanidine hydrochloride, and calcein.

4: The method of claim 1, wherein the amplifying is a reverse-transcription loop-mediated isothermal amplification (RT-LAMP) process.

5: The method of claim 1, further comprising:
extracting nucleic acid from the sample.

6: The method of claim 1, wherein the assay is a colorimetric RT-LAMP assay.

7: The method of claim 6, wherein the assaying comprises:
mixing a master mix, a primer mix, the sample, calcein, and dH₂O to form a colorimetric RT-LAMP mixture; and
incubating the colorimetric RT-LAMP mixture in a water bath at a constant temperature for a time.

8: The method of claim 7, wherein incubating the colorimetric RT-LAMP mixture occurs for 20 to 70 minutes.

9: The method of claim 1, wherein the primer set is N-ID5 and a limit of detection of the assay is 0.01 to 0.2 copies/μL of the sample.

10: The method of claim 1, wherein the SARS-CoV-2 is SARS-CoV-2, and wherein the SARS-CoV-2 comprises at least one variant selected from the following group consisting of alpha (B.1.1.7), beta (B.1.351), gamma (P.1), delta (B.1.617.2), and omicron (B.1.1.529).

11: The method of claim 1, wherein the target sequence of the SARS-CoV-2 nucleic acid sequence is located within at least one gene selected from the group consisting of nucleocapsid (N), spike (S), RNA-dependent RNA polymerase (RdRp), and envelope (E) of a SARS-CoV-2 genome.

12: The method of claim 1, wherein detecting the presence of the amplified target sequence is observed with a visual color change, a transilluminator, and an ultraviolet light.

13: The method of claim 1, wherein an average time to react (TTR) is from 20 to 60 minutes.

14: The method of claim 1, wherein the primer set is N-ID5, and the detection of the presence of the amplification product for the target sequence of a SARS-CoV-2 nucleic acid sequence includes an accuracy of at least 94% for the colorimetric RT-LAMP assay, wherein accuracy is based on colorimetric RT-LAMP assay results compared to reverse transcription real-time polymerase chain reaction (RT-qPCR) assay results of the sample with a sample size of at least 65 samples.

15: The method of claim 1, wherein the primer set is N-ID5, and the assay has a specificity of at least 99% for the colorimetric RT-LAMP assay, wherein specificity is based on colorimetric RT-LAMP assay results compared to RT-qPCR assay results of the sample with a sample size of at least 65 samples.

16: The method of claim 1, wherein the primer set is N-ID5, and the assay has a sensitivity of at least 90% for the colorimetric RT-LAMP assay, wherein sensitivity is based on colorimetric RT-LAMP assay results compared to RT-qPCR assay results of the sample with a sample size of at least 65 samples.

17: The method of claim 1, wherein the primer set is N-ID5 and a positive-percent agreement of the RT-LAMP assay of the sample is at least 99% based on a true positive and a false positive when compared to real-time reverse-transcriptase polymerase chain reaction (RT-qPCR) assay results of the sample with a sample size of at least 65 samples.

18: The method of claim 1, wherein the primer set is N-ID5 and a negative-percent agreement of the RT-LAMP assay of the sample is at least 85% based on a true negative and a false negative when compared to real-time reverse-transcriptase polymerase chain reaction (RT-qPCR) assay results of the sample with a sample size of at least 65 samples.

19: The method of claim 1, wherein the method further comprises treating a subject for which the sample was obtained.

20: A kit for detection of SARS-CoV-2 in a sample, the kit comprising:
a reverse transcriptase, a polymerase, calcein, a universal primer set for loop-mediated isothermal amplification (LAMP) of a target sequence in a SARS-CoV-2 nucleic acid sequence and variants thereof containing mutations within one or more primer binding sites;
wherein the universal primer set suitable for LAMP hybridizes to the target sequence of a SARS-CoV-2 nucleic acid sequence in the presence of a plurality of undefined mutations, and is configured to provide a positive result for the target sequence of a SARS-CoV-2 nucleic acid sequence in a predetermined assay time period otherwise determined for a positive sample of a target nucleic acid having a known sequence;
wherein any of the reagents in the kit may be combined in a mixture in a single container or provided in separate containers.
